(19) United States
(12) Patent Application Publication
(10) Pub. No.: US 2024/0390905 A1
(43) Pub. Date: Nov. 28, 2024

(54) DEVICES AND METHODS FOR ELECTROMAGNETIC SENSING OF LATERAL FLOW ASSAYS

(71) Applicant: 11281232 CANADA INC., Montreal (CA)

(72) Inventors: Patrick O'Neill, Montreal (CA); Adam Melnyk, Toronto (CA); Mark Kumhyr, Kanata (CA)

(21) Appl. No.: 18/705,013

(22) PCT Filed: Nov. 17, 2022
(86) PCT No.: PCT/IB2022/061073

§ 371 Date: Apr. 26, 2024

Related U.S. Application Data
(60) Provisional application No. 63/280,191, filed on Nov. 17, 2021.

(51) Int. Cl. B01L 3/00; G01N 27/22; G01N 27/74

(52) U.S. Cl. CPC B01L 3/502761; B01L 3/5023; G01N 27/22; G01N 27/74; B01L 2200/0647; B01L 2300/0645; B01L 2300/069; B01L 2300/0825

(57) ABSTRACT

There is provided a testing device for evaluation of at least one analyte, comprising: a planar element, at least one electronic sensor disposed on the planar element for sensing at least one sensing region of a lateral flow test element for evaluation of the at least one analyte, and an adhesive substrate connected to a surface of the planar element and set to connect to a surface of the lateral flow test element.

### RELATED APPLICATION

This application claims the benefit of priority of U.S. Provisional Patent Application No. 63/280,191 filed on Nov. 17, 2021, the contents of which are incorporated herein by reference in their entirety.

### FIELD AND BACKGROUND OF THE INVENTION

The present invention, in some embodiments thereof, relates to medical diagnostic devices and, more specifically, but not exclusively, to lateral flow based medical devices.

Lateral flow assays (LFAs) are ubiquitous in the field of rapid diagnostics, with the most commonplace application of the technology being the pregnancy test. Today, LFAs are also capable of detecting infectious diseases such as COVID-19 and biomarkers like prostate-specific antigen.

### SUMMARY OF THE INVENTION

According to a first aspect, a testing device for evaluation of at least one analyte, comprises:
a planar element, at least one electronic sensor disposed on the planar element for sensing at least one sensing region of a lateral flow test element for evaluation of the at least one analyte, and an adhesive substrate connected to a surface of the planar element and set to connect to a surface of the lateral flow test element.

In a further implementation form of the first aspect, the adhesive substrate is spread over the surface of the planar element and set to spread over the surface of the lateral flow test element.

In a further implementation form of the first aspect, the adhesive substrate comprises a first and second adhesive opposing surfaces, the first adhesive surface connected to the surface of the planar element, the second adhesive surface set to connect to the surface of the lateral flow test element.

In a further implementation form of the first aspect, the adhesive substrate is selected from a group consisting of: glue, tape, a self-adhesive, and a spray-on adhesive.

In a further implementation form of the first aspect, further comprising the lateral flow test element.

In a further implementation form of the first aspect, the adhesive substrate places the at least one sensing region(s) in close proximity to the at least one electronic sensor.

In a further implementation form of the first aspect, the distribution of the adhesive substrate is selected to exclude a significant amount of air from being present between the at least one sensing region(s) and the at least one electronic sensor.

In a further implementation form of the first aspect, the adhesive substrate is sandwiched between the surface of the planar element and the surface of the lateral flow test element.

In a further implementation form of the first aspect, the adhesive substrate is sandwiched between the at least one electronic sensor and a region of the lateral test flow element that includes the at least one sensing region.

In a further implementation form of the first aspect, the adhesive substrate is disposed at least over the at least one electronic sensor and the at least one sensing region, wherein a region of the lateral test flow element that includes the at least one sensing region is aligned and facing the at least one electronic sensor.

In a further implementation form of the first aspect, the at least one sensing region of the lateral flow test element includes analyte binding elements designed to bind to the at least one analyte administered to the lateral flow test element.

In a further implementation form of the first aspect, the planar element replaces a backing card of the lateral flow test element that otherwise serves as a physical support component of the lateral flow test element.

In a further implementation form of the first aspect, a thickness of the adhesive substrate is selected to be thin for obtaining a sufficiently strong signal above a threshold from the at least one electronic sensor sensing the at least one sensing region for evaluation of the at least one analyte.

In a further implementation form of the first aspect, the surface of the planar element has a size of at least the size of the surface of the lateral flow test element.

In a further implementation form of the first aspect, the planar element comprises a printed circuit board (PCB), wherein the at least one electronic sensor is at least one of: connected to and integrated within the PCB.

In a further implementation form of the first aspect, further comprising at least one second electronic sensor disposed on the planar element for sensing at least one reference region of the lateral flow test that does not substantially include analyte binding elements, wherein the at least one analyte administered to the lateral flow test element flows across the at least one reference region without substantially selectively binding thereto.

In a further implementation form of the first aspect, signals outputted by the at least one second electronic sensor sensing the at least one second region are analyzed to help measure the at least one analyte.

In a further implementation form of the first aspect, the at least one sensing region comprises a testing region and a reference region distinct from the testing region, wherein the at least one electronic sensor comprises a test electronic sensor located on the planar element for sensing the testing region, and a reference electronic sensor located on the planar element for sensing the reference region.

In a further implementation form of the first aspect, the at least one sensing region includes analyte binding elements designed to bind to the at least one analyte corresponding to infectious disease biomarkers for evaluation of presence of the infectious disease in a user.

In a further implementation form of the first aspect, the infectious disease biomarkers comprise markers for at least one of: a sexually transmitted infection and COVID-19.

In a further implementation form of the first aspect, the at least one sensing region includes analyte binding elements designed to bind to the at least one analyte corresponding to health-related biomarkers for evaluation of relevant health conditions in a user.

In a further implementation form of the first aspect, the health-related biomarkers are selected from a group consisting of: cortisol, melatonin, testosterone, and progesterone.

In a further implementation form of the first aspect, the at least one electronic sensor is designed to generate electromagnetic fields that follow field lines that originate from the at least one electronic sensor, pass through the adhesive substrate, pass through the at least one sensing region, pass back through the adhesive substrate, and back to the at least one electronic sensor.

In a further implementation form of the first aspect, the at least one electronic sensor comprises at least one capacitive sensor for capacitive sensing.

In a further implementation form of the first aspect, each capacitive sensor comprises an interdigitated arrangement of a pair of electrodes.

In a further implementation form of the first aspect, the at least one electronic sensor comprises at least one inductive sensor for inductive sensing.

In a further implementation form of the first aspect, each inductive sensor comprises at least one planar coil with at least one layer.

In a further implementation form of the first aspect, further comprising circuitry configured to activate the at least one inductive sensor at a resonant frequency thereof.

In a further implementation form of the first aspect, further comprising a controller operating at a sampling frequency and configured to measure changes in the resonant frequency of the at least one electronic sensor indicating binding of the at least one analyte to the at least one sensing region.

In a further implementation form of the first aspect, the resonant frequency of the at least one electronic sensor is in a range of 0.001-40 megahertz (MHz).

In a further implementation form of the first aspect, a label selected to exhibit superparamagnetic properties is used to identify the presence of the at least one analyte.

In a further implementation form of the first aspect, further comprising a transceiver configured to transmit signals sensed by the at least one electronic sensor to an external computing device for analysis of the signals for measuring presence of the at least one analyte.

In a further implementation form of the first aspect, the testing device is implemented as a single-use device designed to be disposed of after a liquid sample is applied to the lateral flow test element, the liquid sample flows through the lateral flow test element by capillary action and reaches a wick, and the at least one analyte when present in the solution binds to analyte binding elements of the at least one sensing region.

According to a second aspect, a computer implemented method for evaluation of at least one analyte, comprises: receiving signals obtained from at least one electronic sensor of a testing device, wherein the at least one electronic sensor is disposed on a planar element of the testing device for sensing at least one sensing region of a lateral flow test element, the at least one sensing region includes analyte binding elements designed to bind to the at least one analyte administered to the lateral flow test element, and an adhesive substrate is connected to a surface of the planar element and to a surface of the lateral flow test element for fixing the at least one electronic sensor in a position facing a region of the lateral flow test element that includes the at least one sensing region, analyzing the signals for measuring accumulation of the at least one analyte at the at least one sensing region of the lateral flow test, and determining at least one of: evidence indicating the presence of the at least one analyte, concentration of the at least one analyte, and evidence indicating lack of presence of the at least one analyte.

In a further implementation form of the second aspect, the at least one electronic sensor is selected from a group consisting of at least one capacitive sensor for capacitive sensing, and at least one inductive sensor for inductive sensing.

In a further implementation form of the second aspect, further comprising determining at least one of: a diagnosis, a prognosis, or a trend of a medical condition or an analysis of a wellness state, when at least one of the following is met: the at least one analyte is detected, the at least one analyte is not detected, the concentration of the at least one analyte is above a threshold, and the concentration of the at least one analyte is below the threshold.

In a further implementation form of the second aspect, further comprising in response to the diagnosis, prognosis, or trend, establishing a communication session with a remote client terminal of a healthcare provider and/or public health entity.

In a further implementation form of the second aspect, further comprising administering a treatment to a user that is effective for treating the medical condition.

In a further implementation form of the second aspect, further comprising determining the concentration of a wellness biomarker comprising non-diagnosis information and communicating the non-diagnostic information automatically to a client terminal via a connected smart device.

In a further implementation form of the second aspect, the wellness state is a non-reportable, non-life-threatening health state not requiring diagnosis by a medical professional, categorized by varied biomarker levels.

In a further implementation form of the second aspect, the biomarker is selected from a group consisting of salivary cortisol, blood-based cortisol, testosterone, interleukin-6 (IL-6), tetrahydrocannabinol (THC) influenza A, influenza B, group A streptococcus, procalcitonin, C-reactive protein, and Escherichia coli (E. coli).

In a further implementation form of the second aspect, the medical condition is an infection with an infectious disease agent.

In a further implementation form of the second aspect, the infectious disease agent is selected from a group consisting of: a sexually transmitted infection and COVID-19.

In a further implementation form of the second aspect, analyzing comprises feeding the signals into a machine learning model trained on a training dataset that includes a plurality of records, each record including a sample signal labelled with a ground truth indication of at least one of: evidence of presence of the at least one analyte, evidence of lack of presence of the at least one analyte, and concentration of the at least one analyte.

In a further implementation form of the second aspect, the received signals are computed from signals outputted by a test electronic sensor that senses the at least one sensing region and a reference electronic sensor that senses a reference region distinct and apart from the at least one sensing region, wherein the at least one analyte which binds to the analyte binding elements in the at least one sensing region does not selectively bind to the reference region.

According to a third aspect, a method of manufacturing a test device, comprises: unrolling a plurality of rolls comprising components for creating a plurality of test devices, unrolling a roll of a flexible planar element having a plurality of spaced apart electronic sensors disposed thereon, layering an adhesive substrate over a surface of the flexible planar element, laminating the components for creating the plurality of testing devices to the adhesive substrate to create a master assembly, and slicing the master assembly into a plurality of slices, each slice denoting a respective testing device of the plurality of testing devices for evaluating presence, absence, and/or concentration of at least one analyte in a sample applied thereon.

In a further implementation form of the third aspect, the flexible planar element comprises a flexible printed circuit board (PCB).

In a further implementation form of the third aspect, further comprising unrolling a roll of self-adhesive tape, and layering the unrolled self-adhesive tape over the surface of the flexible planar element, wherein laminating comprises sandwiching the self-adhesive tape between the planar element and the components of the plurality of testing devices.

In a further implementation form of the third aspect, slicing comprises slicing along a width of the master assembly between neighboring electronic sensors.

In a further implementation form of the third aspect, each respective testing device includes a planar element of the roll of flexible planar element, at least one electronic sensor of the plurality of electronic sensors of the roll of flexible planar element, and the adhesive substrate connected to a surface of the planar element and to a surface of the lateral flow test element for fixing the at least one electronic sensor in a position facing the at least one sensing region that includes analyte binding elements designed to bind to the at least one analyte administered to the lateral flow test element.

According to a fourth aspect, a testing device for evaluation of an analyte, comprises: a planar element, at least one electronic sensor disposed on the planar element for sensing at least one sensing region of a lateral flow test element for evaluation of the analyte, and an adhesive substrate connected to a surface of the planar element and set to connect to a surface of the lateral flow test element.

In a further implementation form of the first aspect or the fourth aspect, the adhesive substrate is spread over the surface of the planar element and the surface of the lateral flow test element.

In a further implementation form of the first aspect or the fourth aspect, the adhesive substrate includes an insignificant amount of air that does not significantly impact transmission of signals, wherein an insignificant amount of air is present in the adhesive substrate in continuous contact between the at least one electronic sensor and the at least one sensing region.

In a further implementation form of the first aspect or the fourth aspect, the adhesive substrate comprises an adhesive substrate having a first and second adhesive opposing surfaces, the first adhesive surface connected to the surface of the planar element, the second adhesive surface set to connect to the surface of the lateral flow test element.

In a further implementation form of the first aspect or the fourth aspect, the adhesive substrate comprises glue.

In a further implementation form of the first aspect or the fourth aspect, the adhesive substrate is sandwiched between the surface of the planar element and the surface of the lateral flow test element.

In a further implementation form of the first aspect or the fourth aspect, the adhesive substrate is sandwiched between the at least one electronic sensor and a region of the lateral test flow element that includes the at least one sensing region.

In a further implementation form of the first aspect or the fourth aspect, the adhesive substrate is disposed at least over the at least one electronic sensor and the at least one sensing region, wherein a region of the lateral test flow element that includes the at least one sensing region is aligned and facing the at least one electronic sensor.

In a further implementation form of the first aspect or the fourth aspect, a thickness of the adhesive substrate is selected to be thin for obtaining a sufficiently strong signal above a threshold from the at least one electronic sensor sensing the at least one sensing region for evaluation of the analyte.

In a further implementation form of the first aspect or the fourth aspect, the surface of the planar element has a size of at least the size of the surface of the lateral flow test element.

In a further implementation form of the first aspect or the fourth aspect, the testing device further comprises the lateral flow test element.

In a further implementation form of the first aspect or the fourth aspect, the at least one sensing region of the lateral flow test element includes analyte binding elements designed to bind to the analyte administered to the lateral flow test element.

In a further implementation form of the first aspect or the fourth aspect, the planar element comprises a printed circuit board (PCB), the PCB replacing a backing card of the lateral flow test element, wherein the at least one electronic sensor is at least one of: connected to and integrated within the PCB.

In a further implementation form of the first aspect or the fourth aspect, a backing card of the lateral flow test element is affixed to the planar element, wherein the backing card serves as a physical support component of the lateral flow test element.

In a further implementation form of the first aspect or the fourth aspect, the testing device further comprises at least one second electronic sensor located within the planar element for sensing at least one reference region of the lateral flow test that excludes analyte binding elements, wherein the analyte administered to the lateral flow test element flows across the at least one reference region without selectively binding thereto.

In a further implementation form of the first aspect or the fourth aspect, signals outputted by the at least one second electronic sensor sensing the at least one second region are analyzed to help detect presence of the analyte.

In a further implementation form of the first aspect or the fourth aspect, the at least one sensing region comprises a testing region and a reference region distinct from the testing region, wherein the at least one electronic sensor comprises a test electronic sensor located on the planar element for sensing the testing region, and a reference electronic sensor located on the planar element for sensing the reference region.

In a further implementation form of the first aspect or the fourth aspect, the at least one sensing region includes analyte binding elements designed to bind to the analyte comprising infectious disease biomarkers for evaluation of presence of the infectious disease in a user.

In a further implementation form of the first aspect or the fourth aspect, the infectious disease biomarkers comprise markers for at least one of: sexually transmitted infections, and COVID-19.

In a further implementation form of the first aspect or the fourth aspect, the at least one electronic sensor is designed to generate electromagnetic fields that follow field lines that originate from the at least one electronic sensor, pass through the adhesive substrate, pass through the at least one sensing region, pass back through the adhesive substrate, and back to the at least one electronic sensor.

In a further implementation form of the first aspect or the fourth aspect, the at least one electronic sensor comprises at least one capacitive sensor for capacitive sensing.

In a further implementation form of the first aspect or the fourth aspect, each capacitive sensor comprises an interdigitated arrangement of a pair of electrodes.

In a further implementation form of the first aspect or the fourth aspect, the at least one electronic sensor comprises at least one inductive sensor for inductive sensing.

In a further implementation form of the first aspect or the fourth aspect, each inductive sensor comprises at least one planar coil with at least one layer.

In a further implementation form of the first aspect or the fourth aspect, the testing device further comprises circuitry configured to activate the at least one inductive sensor at a resonant frequency thereof.

In a further implementation form of the first aspect or the fourth aspect, the testing device further comprises a controller operating at a sampling frequency and configured to measure changes in the resonant frequency of the at least one electronic sensor indicating binding of analytes to the at least one sensing region.

In a further implementation form of the first aspect or the fourth aspect, the resonant frequency of the at least one electronic sensor is in a range of 1-10 megahertz (MHz).

In a further implementation form of the first aspect or the fourth aspect, a label selected to exhibit superparamagnetic properties is used to identify the presence of the analyte.

In a further implementation form of the first aspect or the fourth aspect, the testing device further comprises a transceiver configured to transmit signals sensed by the at least one electronic sensor to an external computing device for analysis of the signals for detecting presence of the analyte.

In a further implementation form of the first aspect or the fourth aspect, the testing device is a single-use device designed to be disposed of after a liquid sample is applied to the lateral flow test element, the liquid sample flows through the lateral flow test element by capillary action and reaches a wick, and the analyte when present in the solution binds to analyte binding elements of the at least one sensing region.

According to a fifth aspect, a computer implemented method for evaluation of an analyte, comprises: receiving signals obtained from at least one electronic sensor of a testing device, wherein the at least one electronic sensor is selected from a group consisting of at least one capacitive sensor for capacitive sensing, and at least one inductive sensor for inductive sensing, wherein the at least one electronic sensor is disposed on a planar element of the testing device for sensing at least one sensing region of a lateral flow test element, the at least one testing region includes analyte binding elements designed to bind to the analyte administered to the lateral flow test element, and an adhesive substrate is connected to a surface of the planar element and to a surface of the lateral flow test element for fixing the at least one electronic sensor in a position facing a region of the lateral flow test element that includes the at least one sensing region, analyzing the signals for measuring accumulation of the analyte at the at least one sensing region of the lateral flow test, and determining at least one of: the presence of the analyte, concentration of the analyte, and no evidence of presence of the analyte.

In a further implementation form of the second aspect or fifth aspect, the method further comprises determining a diagnosis or prognosis of a medical condition or wellness state when at least one of the following is met: the analyte is present, the analyte is absent, the concentration of the analyte is above a threshold, and the concentration of the analyte is below the threshold.

In a further implementation form of the second aspect or fifth aspect, the medical condition is an infection with an infectious disease agent.

In a further implementation form of the second aspect or fifth aspect, the infectious disease agent is selected from a group consisting of COVID-19, and a sexually transmitted infection, a respiratory infection, a hormone marker indicative of a wellness state, a protein marker indicative of an infectious disease or wellness state, a nucleic acid-based marker indicative of an infectious disease or a wellness state, and a marker indicating the use of a drug of abuse.

In a further implementation form of the second aspect or fifth aspect, the liquid sample is selected from a group consisting of blood, saliva, urine, and sweat.

In a further implementation form of the second aspect or fifth aspect, the method further comprises, in response to the diagnosis, establishing a communication session with a remote client terminal of a healthcare provider and/or public health entity.

In a further implementation form of the second aspect or fifth aspect, the method further comprises administering a treatment to a user that is effective for treating the medical condition.

In a further implementation form of the second aspect or fifth aspect, treatment can be understood as referring to both medical (drugs, therapies, etc.) and non-medical (diet, exercise, supplements, habits) types of treatment.

In a further implementation form of the second aspect or fifth aspect, analyzing comprises feeding the signals into a machine learning model trained on a training dataset that includes a plurality of records, each record including a sample signal labelled with a ground truth indication of at least one of: evidence for presence of the analyte, no evidence of presence of the analyte, and concentration of the analyte.

In a further implementation form of the second aspect or fifth aspect, the received signals are computed from signals outputted by a test electronic sensor that senses the at least one testing region and a reference electronic sensor that senses a reference region distinct and apart from the at least one testing region, wherein the analyte which binds to the analyte binding elements in the at least one sensing region does not selectively bind to the reference region.

According to a sixth aspect, a method of manufacturing a lateral flow test device, comprises: unrolling a plurality of rolls comprising components for creating a plurality of lateral flow test elements, unrolling a roll of a flexible planar element having a plurality of spaced apart electronic sensors disposed thereon, layering an adhesive substrate over a surface of the flexible planar element, laminating the components for creating the plurality of lateral flow test elements to the adhesive substrate to create a master assembly, and slicing the master assembly into a plurality of slices, each slice denoting a respective testing device for evaluating presence, no evidence of presence, and/or concentration of an analyte in a sample applied thereon.

In a further implementation form of the third aspect or sixth aspect, the flexible planar element comprises a flexible printed circuit board (PCB).

In a further implementation form of the third aspect or sixth aspect, the method further comprises unrolling a roll of self-adhesive tape, and layering comprises layering the unrolled self-adhesive tape over the surface of the flexible planar element.

In a further implementation form of the third aspect or sixth aspect, wherein slicing comprises slicing along a width of the master assembly between neighboring electronic sensors.

In a further implementation form of the third aspect or sixth aspect, each respective testing device includes a respective lateral flow test element of the plurality of lateral flow test elements, a planar element of the roll of flexible planar element, at least one electronic sensor of the plurality of electronic sensors of the roll of flexible planar element, and the adhesive substrate connected to a surface of the planar element and to a surface of the lateral flow test element for fixing the at least one electronic sensor in a position facing the at least one sensing region that includes analyte binding elements designed to bind to the analyte administered to the lateral flow test element.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system for evaluating an analyte, in accordance with some embodiments of the present invention;

FIG. 2 is a schematic of components for assembling an exemplary testing device for evaluation of an analyte, in accordance with some embodiments of the present invention;

FIG. 3 is a schematic depicting the process of assembling the testing device, from components as described with reference to FIG. 2, in accordance with some embodiments of the present invention;

FIG. 4A is a schematic of electric field lines passing between sensors of the planar element of the testing device, in accordance with some embodiments of the present invention;

FIG. 4B is a schematic of magnetic field lines passing through sensors of the planar element of the testing device, in accordance with some embodiments of the present invention;

FIG. 5 is a schematic of an assembled testing device, shown in a perspective view and cross sectional view in accordance with some embodiments of the present invention:

FIG. 6 is a schematic depicting application of a sample to the testing device, in accordance with some embodiments described herein;

FIG. 7 is a schematic depicting a state of the diagnostic testing device at the end of its lifecycle, i.e., when the liquid has traveled through the multiple membrane components of an LFA and into a wick, in accordance with some embodiments of the present invention;

FIG. 8 is a schematic of an exemplary process for analyzing signals obtained from a testing device, in accordance with some embodiments of the present invention;

FIG. 9 is a schematic depicting a perspective view of roll-to-roll (R2R) manufacturing of testing devices that include LFA connected to a PCB via an adhesive substrate, in accordance with some embodiments of the present invention;

FIG. 10 is a flowchart of an exemplary process of R2R manufacturing of a testing device, in accordance with some embodiments of the present invention;

FIG. 11 is a schematic depicting an exemplary GUI presented on a display of a smartphone, presenting instructions for using and/or testing results, of a testing device that includes a lateral flow assay connected via adhesive tape with PCB that includes sensors, in accordance with some embodiments of the present invention;

FIG. 12A is a schematic of an exemplary planar element implemented as a PCB comprising a resonant magnetometer designed to connect to a LFA via adhesive substrate, in accordance with some embodiments of the present invention;

FIG. 12B is a schematic of an exemplary planar element implemented as a PCB designed to connect to a LFA via adhesive substrate and interchangeably connect to an exemplary resonant magnetometer, in accordance with some embodiments of the present invention;

FIG. 13 is a graph depicting a response curve from a reference coil and a response curve from a test coil over time, as measured by inventors, in accordance with some embodiments of the invention;

FIG. 14 is a graph depicting the phenomenon of ions flowing across coils creating changes to the resonant frequency of the coils and the calculated inductance, as measured by inventors, in accordance with some embodiments of the invention;

FIG. 15 is a continuation of curves from the graph of FIG. 13, extending for a longer amount of time, in accordance with some embodiments of the present invention;

FIG. 16 is a graph computed by subtracting normalized inductance of the test coil from the normalized inductance of the reference coil after running the test for long enough that the inductances have stabilized, in accordance with some embodiments of the present invention;

FIG. 17 is a graph illustrating the difference in the dynamic response between a positive curve and a negative curve of the electromagnetic sensing (EMS) modality used in a testing device fabricated by Inventors, in accordance with some embodiments of the present invention;

FIG. 18 is a graph comparing electromagnetic data measured using embodiments described herein, to colorimetric data measured using standard optical approaches, illustrating the advantage electromagnetic sensing (EMS) has over optical sensing methods currently used in the lateral flow industry;

FIG. 19 is a schematic of an exemplary inductive sensor configuration and immunosensor assembly used in an experiment for testing of magnetic IL-6 lateral flow strips, in accordance with some embodiments of the present invention;

FIG. 20 includes standard curves presenting the effect of IL-6 concentration on signal readout of two inductive immunosensor compensation schemes used in the experiment that evaluated the testing of magnetic IL-6 lateral flow strips, in accordance with some embodiments of the present invention;

FIG. 21 includes time series signal profiles of two inductive immunosensor compensation schemes for a test run at 10 ng/mL of IL-6, of the IL-6 experiment, in accordance with some embodiments of the present invention;

FIG. 22 depicts averaged truncated time series signal profiles of two inductive immunosensor compensation schemes for all of the tests run in the IL-6 experiment, in accordance with some embodiments of the present invention; and

FIG. 23 includes schematics depicting top views of schematics 604A-D described with reference to FIG. 6 and corresponding sensor response curves, in accordance with some embodiments described herein.

### DESCRIPTION OF SOME EMBODIMENTS OF THE INVENTION

The present invention, in some embodiments thereof, relates to a medical device and, more specifically, but not exclusively, to lateral flow assay based medical devices.

The term analyte binding elements as used herein, refers to any biological or non-biological reagent that shares a strong and selective affinity with a specific analyte. Examples of analyte binding elements include, but are not limited to antibodies, recombinant proteins, peptides, enzymes, aptamers, nucleic acids, and molecularly imprinted polymers. The analyte binding elements may selectively bind to the specific analyte upon interacting with one another.

An aspect of some embodiments of the present invention relates to a testing device for evaluation of an analyte. The analyte, when present, is within a liquid sample applied to a lateral flow test element component connected to the testing device. The testing device includes one or more electronic sensors (sometimes referred to herein as “sensors”) located on a planar element. The sensors are set for sensing one or more sensing regions of a lateral flow test element, for evaluation of the analyte, for example, signals outputted by the sensors are analyzed to determine presence and/or concentration of the analyte at the sensing region(s). An adhesive substrate is connected to a surface of the planar element and set to connect to a surface of the lateral flow test element. The adhesive substrate fixes the sensor(s) in position relative to the sensing region(s) of the lateral flow test element. When the adhesive substrate is sandwiched between the sensor(s) and the sensing region(s), the adhesive substrate is selected to have a thickness and/or to be made of a material that enables the sensor(s) to sense the sensing region(s) with a signal strength sufficient to obtain an accurate measurement (e.g., signal strength above a threshold, probability of measurement made based on the signals above a threshold).

The testing device optionally includes the lateral flow test element. The lateral flow test element may be a stand in for lateral flow tests, microfluidics, microarrays, or any other testing format with a sensing region along a substantially planar interface.

It is noted that the sensing region described herein is an area on the lateral flow test element that is being probed by the electronic sensor(s). The sensing region may include test region(s) (i.e., where analyte binding elements are present) and/or may include reference region(s).

In some embodiments, the testing device includes the planar element with sensor(s), and the adhesive substrate that is set to connect to a surface of the lateral flow test element, before the lateral flow test element is connected. In other implementations, also referred to herein as testing device assemblies, the testing device includes the assembly of the planar element with sensor(s), the adhesive substrate, and the lateral flow test element.

The testing device described herein may be an all-in-one system, or one part of a multi-part system, for example, a two-part system that includes a cartridge (i.e., the testing device) and reading device designed to collect test signals from the cartridge. The term cartridge may be interchanged with the term testing device. The all-in-one and multi-part systems may additionally communicate with a nearby connected smart device (e.g., smartphone), for example, to analyze data collected from the testing device and/or display interactive test results from the testing device to a user. In the multi-part system, optionally the two-part system, the user may reuse at least one component of the system (e.g., a reading device) with different disposable cartridges. In some embodiments of the two-part system comprising a reading device, a disposable cartridge is interchangeably connected (e.g., by insertion) to the reading device, and the reading device collects signals from the sensors on the disposable cartridge. The reading device may additionally transmit the signals to a nearby connected smart device. In some embodiments of the all-in-one system, the testing device may directly communicate with the connected smart device such that the user does not require a reading device to receive test results.

An aspect of some embodiments of the present invention relates to systems, methods, devices, and/or code instructions (e.g., stored on a data storage device and executable by one or more hardware processors) for evaluation of an analyte. Signals are obtained from sensor(s) of a testing device to which a liquid sample, potentially including an analyte, is applied. Exemplary sensors include a capacitive sensor(s) for capacitive sensing, and/or an inductive sensor(s) for inductive sensing. The sensor(s) is disposed on, within, and/or around a planar element of the testing device (e.g., embedded into the planar element, placed on top of the planar element) for sensing one or more sensing regions of a lateral flow test element to which the liquid sample has been applied. The sensing region(s) may include analyte binding elements designed to bind to the analyte within the liquid sample. The term testing region(s) as used herein, refers to the sensing region(s) that include the analyte binding elements. The term reference region(s) as used herein, refers to the portion of the sensing region(s) not intended to bind the analyte. Signals obtained from the testing region(s) may be analyzed with reference to the reference region(s), as described herein. An adhesive substrate is connected to a surface of the planar element and to a surface of the lateral flow test element for fixing the sensor(s) in a position facing a region of the lateral flow test element that includes the testing region(s) and reference region(s) of the sensing region(s). The signals are analyzed for measuring selective binding and accumulation of the analyte at the testing region(s) of the lateral flow test. The evidence of presence of the analyte, the amount of the analyte bound, lack of evidence of presence of the analyte, and/or concentration of the analyte, is determined according to the analysis. A diagnosis and/or prognosis of a medical condition, for example, infection with an infectious disease agent may be made according to the detected evidence of presence of the analyte, lack of evidence presence of the analyte, and/or concentration of the analyte. The result may be presented on a computing device, for example, a smartphone. This produces clear accurate results, and avoids potential errors that might otherwise occur during the interpretation of standard lateral flow assays, such as a user waiting too long and reading a positive outcome when the outcome is actually negative, or missing a faint line thereby reading a positive outcome as negative. The results may be, for example, a health-related condition, wellness state, or generalized analyte concentration in a sample (e.g. bacteria for evaluation of food safety). The wellness state may be, for example, a non-reportable, non-life-threatening health state not requiring diagnosis by a medical professional, categorized by varied biomarker levels.

The adhesive described herein ensures the proper alignment of the planar element and sensors with respect to the lateral flow test element after assembly of the testing device (i.e., joining of the planar element to the lateral flow test element via the adhesive).

An aspect of some embodiments of the present invention relates to a method for manufacturing of the test device described herein. The test device may be manufactured using a modification of a roll-to-roll (R2R) manufacturing method (also referred to as reel-to-reel manufacturing). Rolls of components for creating multiple lateral flow test elements are unrolled. A roll of a flexible planar element is unrolled. The roll of the flexible planar element has multiple spaced apart sensors disposed thereon. The roll of the flexible planar element may be a flexible printed circuit board (PCB) that integrates and/or connects to the sensor(s). An adhesive substrate is layered over a surface of the flexible board. The adhesive substrate may be, for example, a roll of self-adhesive tape that is unrolled, a spray-on adhesive, and/or a glue that is sprayed by a sprayer and/or applied by an applicator. The components for creating the lateral flow test elements are laminated to the adhesive substrate to create a master assembly. The master assembly is sliced into multiple slices. Each slice denotes a respective test device for testing for an analyte in a sample applied thereon, as described herein.

At least some embodiments described herein address the technical problem of improving accuracy of measuring an analyte, and/or improving usability of a test device, optionally a lateral flow test device, that measures an analyte. At least some embodiments described herein improve the technical field of lateral flow testing devices, by increasing accuracy of measuring the analyte, and/or increasing usability of the test device (e.g., lateral flow test device). In at least some embodiments, a solution to the technical problem, and/or the improvement to the technical field, is provided by electronic sensor(s) located on the planar element of the testing device, and the adhesive substrate that is applied to the surface of the planar element for consistency in connecting a region of the lateral flow test element that includes the sensing region facing the electronic sensor. The adhesive substrate fixes the position of the sensor(s) in close proximity to the sensing region(s), improving accuracy of the measured signals, for example, enabling detection of low concentrations of the analyte. When the adhesive substrate is sandwiched between the electronic sensor(s) and a region of the lateral test flow element that includes the sensing region(s), the height (e.g., thinness) of the adhesive substrate is selected to enable sufficient signal strength of the sensor(s) sensing the sensing region(s) through the adhesive substrate, which improves sensitivity and/or signal to noise ratio of the testing device. The height of the adhesive substrate may be minimized. The improvement is in contrast, for example, to colorimetric lateral flow assays that use visual indicators to indicate evidence of presence of the analyte, or lack of evidence of presence of the analyte. Such lateral flow assays effectively provide a binary outcome when they are interpreted with the naked eye, indicating evidence of presence of the analyte or lack of evidence of presence of the analyte, which makes it difficult to determine concentration and/or detect concentrations of the analyte exhibiting signals below the visual threshold. i.e. resolvable by eye or with a camera. The sensors, set in position by the adhesive substrate, collect reliable signals that are translated into clear, accurate results for the user, and avoids potential errors that might otherwise occur in standard lateral flow assays, such as the user waiting too long and reading a positive outcome when the outcome is actually negative, or missing a faint line thereby reading a positive outcome as negative.

The performance of electronic measuring devices such as capacitive or inductive based sensors decreases with increasing distance between the sensing elements and the object and/or environment being probed. At least some implementations described herein include the adhesive substrate for reducing the distance between the lateral flow assay and the printed circuit board (PCB) housing the electronic sensors which sense the sensing regions(s) of the lateral flow test element (e.g., probe the flow and/or accumulation of nanoparticles (e.g., at the test region)), as described herein.

The adhesive described herein ensures the proper alignment of the planar element and sensors with respect to the lateral flow test element after assembly of the testing device (i.e., joining of the planar element to the lateral flow test element via the adhesive).

At least some embodiments described herein address the technical problem of manufacturing the test element described herein. At least some embodiments described herein improve the technology of manufacturing test elements. In at least some embodiments, a solution to the technical problem, and/or the improvement to the technical field, is provided by modifying a roll-to-roll manufacturing approach, by using rolls of a flexible planar element having multiple spaced apart electronic sensors disposed thereon, that is unrolled. The flexible planar element may be a flexible PCB. An adhesive substrate is applied over a surface of the flexible planar boards. The adhesive substrate may be a roll of self-adhesive tape that is unrolled over the surface of the flexible planar element. Unrolled rolls of components for creating multiple lateral flow test elements are laminated to the adhesive substrate to create a master assembly. The master assembly is then sliced to create individual testing devices. This manufacturing method quickly and efficiently creates a large number of testing devices.

At least some implementations of the systems, methods, devices, and/or code instructions described herein address the technical problem of improving accuracy and/or improving ease of use of LFAs and/or other point-of-care (POC) testing devices. At least some implementations of the systems, method, devices, and/or code instructions described herein improve the technology of LFAs and/or POC testing devices, by improving accuracy and/or ease of use.

LFAs are ubiquitous in the field of rapid diagnostics, with the most commonplace application of the technology being the pregnancy test. Today. LFAs are also used to detect infectious diseases such as COVID-19 and biomarkers like prostate-specific antigen. Though versatile, LFAs present several shortcomings, specifically surrounding interpretation and communication of results to the user (e.g., patient). Traditionally, the interpretation of LFA results is done qualitatively via visual signal readouts, such as the presence of a line or dot which can be resolved by eye after the test has had a chance to run, which typically takes between 10 and 15 minutes. This interpretation method presents challenges, for example, for patients who are visually impaired, and also makes it difficult to have quantitative results about the concentration of the analyte in question. Additionally, false negative results can occur if the analyte concentration is not high enough to produce a visual readout observable to the patient, and false positives if the test is interpreted too long after the sample was applied.

In at least some implementations, the technical problem(s) of LFAs are addressed, and/or improvement to the technology of LFAs is provided by, using electronic sensors, optionally performing continuous (or near continuous) electromagnetic sensing (e.g., capacitive, inductive), that are fixed in place by an adhesive substrate for sensing one or more sensing regions. The sensors and/or other electronic components may interface with new and/or existing LFAs, transforming LFAs from qualitative physical tests into quantitative, digitally-integrated platforms. The sensors and/or associated electronic components that are fixed in location for sensing one or more sensing regions by the adhesive substrate enable one or more of:
Detecting lower concentrations of analytes, which may not necessarily produce a detectable signal readout in standard LFAs.
Quantitative measurement of the amount of analyte (e.g., concentration) in the sample, in contrast to standard LFAs that typically provide a binary result, i.e., positive or negative.
Faster time to results using continuous (or near continuous) data monitoring that allows a positive result to be communicated as soon as there is a statistically significant signal arising from the assay. This enablement is in contrast to standard LFAs where a fixed wait time is required before results are available.
Electronic results are available, which may be, for example, presented on a display of a client terminal, stored for future access (e.g., to obtain a trend, such as to detect an increase or change in biomarkers levels over time which may indicate emergence of a medical condition), and/or transmitted electronically to others, for example, to healthcare providers. This enablement is in contrast to standard LFAs where results are only visually indicated on the physical LFA itself. In some LFAs, results are only available for a short time window, where results viewed after the window are invalid.
Testing routinely done in a lab, and/or by trained professionals may be performed at home. In one example, for sexually transmitted infections (STIs) such as chlamydia and gonorrhea. In other examples, testing for other infectious diseases or biomarkers may be performed. In yet another example, at least one of cortisol and melatonin for stress and/or sleep monitoring. In yet another example, levels of bacteria in food may be checked to ensure safe consumption.

At least some implementations of the systems, methods, testing device, and/or code instructions described herein address the technical problem of improving sensitivity, limit of detection (LOD), signal to noise ratio (SNR), and/or dynamic range of sensor based lateral flow assays, for example, capacitive and/or inductive based sensors (e.g., as described herein). At least some implementations of the systems, methods, devices, and/or code instructions described improve the technical field of sensor-based lateral flow assays.

At least some implementations of the systems, methods, devices, and/or code instructions described herein address the technical problem of providing rapid, simple testing for medical conditions, for example, infectious diseases, which may be performed by users themselves, such as in a home setting. At least some implementations of the systems, method, devices, and/or code instructions described herein improve the technology of tests for infectious diseases and biomarkers. An example of an infectious disease is a sexually transmitted infection (STI) such as chlamydia and gonorrhea. Other infectious diseases and/or biomarkers (e.g., proteins, nucleic acids, hormones, vitamins, bacteria, or viruses) may include those present in food, water, feces, urine, saliva, blood, and the like.

At least some implementations of the systems, methods, devices, and/or code instructions described herein enable widespread infectious disease testing by offering a reliable and inexpensive at-home testing solution, and further optional private and convenient access to telemedicine services. At least some implementations enable getting tested, diagnosed, and treated for readily curable infectious diseases (e.g., STIs) all in the same day while ensuring the quality and reliability people expect from medical services. At least some implementations described herein enable measuring health-related biomarkers (e.g., cortisol, melatonin, testosterone, progesterone, tetrahydrocannabinol (THC), influenza A, influenza B, group A streptococcus, procalcitonin. C-reactive protein, and Escherichia coli (E. coli)) at home to provide insights regarding aspects of health and wellness.

At least some implementations described herein improve over other approaches for testing, such as for infectious diseases. Standard approaches to testing include getting tested at a physician's office, at a laboratory, and/or through send-in testing services. Such standard approaches can take 1-3 weeks to receive results, are costly, require trained personnel to perform, and/or are inconvenient for patients. In contrast, at least some implementations described herein provide results in about 5-15 minutes and may be used privately by the patients themselves, such as in their own home.

At least some implementations described herein provide an all-in-one testing solution that augments traditional point-of-care (POC) tests with the addition of a proprietary electronic reader embedded into a single disposable casing.

At least some implementations described herein provide a two-part system, that includes a disposable component (i.e., the testing device described herein) and a reusable reader component designed to obtain measurements from the disposable component.

At least some implementations described herein provide users with an affordable at-home testing solution that allows them to skip the clinic and lengthy time-to-results without compromising their quality of care.

At least some implementations of the systems, methods, devices, and/or code instructions described herein address the technical problem of manufacturing the testing device described herein. At least some implementations of the systems, methods, devices, and/or code instructions described herein improve the process of manufacturing, by providing an approach for manufacturing the testing device described herein.

As used herein, the term lateral flow test strip and lateral flow test element are used interchangeably.

High-throughput manufacturing processes are commonly used for the fabrication of low-cost rapid diagnostics. The LFA is a type of paper-based biodetection technology, which is among the most popular rapid diagnostic formats on the market due in large part to the inexpensive materials and scalable manufacturing processes used to fabricate LFA-based devices. Traditionally, LFAs are manufactured at-scale using a roll-to-roll (R2R) manufacturing process whereby reels of the paper-based membranes and other porous materials of the lateral flow test strip—such as the sample pad, conjugate pad, test or nitrocellulose pad, and wick—are unrolled and sequentially laid onto a reel of backing card. The backing card is generally made of a plastic such as polystyrene (PS) or polyvinyl chloride (PVC) laminated with a pressure-sensitive self-adhesive and serves as the main mechanical support of the LFA. The self-adhesive bonds the backing card and the porous components of the LFA during the R2R manufacturing process.

At least some implementations of the systems, methods, devices, and/or code instructions described provide a scalable approach for manufacturing biosensors as solid-state bioelectric interfaces, which incorporate embedded transduction elements and other electronics directly into ubiquitous biodetection technologies. At least some implementations of the systems, methods, devices, and/or code instructions described provide an approach of R2R manufacturing lateral flow strips in which a reel of flexible PCB performs the function that would otherwise be performed by a standard backing card on which reels of membrane-based lateral flow strip components are assembled, fixed into position using a reel of adhesive tape, and cut into individual strips to create individual testing devices.

In at least some embodiments, the replacement of conventional plastic backing cards of lateral flow strips with flexible PCBs in LFA-based biosensors, as described herein, provides potential advantages and/or technical improvements, for example: (1) embedding the sensors (e.g., transduction elements, planar coils, and/or capacitors) directly into the lateral flow test strip to streamline the manufacturing process, (2) reducing the distance between the sensors (e.g., transduction elements, planar coils, and/or capacitors) and the region of interest on the LFA to improve the sensitivity of the sensor, and (3) combining the backing structure and sensors (e.g., transduction elements, planar coils, and/or capacitors) of the LFA to reduce material costs while maintaining structural integrity and biosensing functionality.

At least some implementations of the systems, methods, devices, and/or code instructions described herein address the technical problem of increasing accuracy, sensitivity, LOD, SNR, and/or dynamic range of lateral flow assays. At least some implementations of the systems, methods, devices, and/or code instructions described herein improve the technology of lateral flow assays, by increasing accuracy, sensitivity, LOD, SNR, and/or dynamic range. In at least some implementations, the technical problem is addressed, and/or the technology is improved, based on electromagnetic sensors that are located within a PCB that is fixed in position relative to a LFA by an adhesive substrate.

At least some implementations of the systems, methods, devices, and/or code instructions described herein address the technical problem of providing simple lateral flow assays designed for use by individuals at home. At least some implementations of the systems, methods, devices, and/or code instructions described herein improve the technology of lateral flow assays by providing simple lateral flow assays designed for use by individuals at home. At least some implementations are based on inductive sensors that are located within a PCB that is fixed in position relative to a LFA by an adhesive substrate. At least some of such implementations provide results, for example, to a mobile device, which may be for example the smartphone of the user.

Magnetic LFAs, unlike their visual or fluorescent counterparts, are generally distinguished by their use of magnetic nanoparticles rather than colloidal gold, latex beads, dyes, or other optical detection labels, though in some embodiments the test is processed in a similar way, with accumulation of particles at the test and control lines representing test results. Magnetic lateral flow readers have the potential to identify the presence of extremely low concentrations of nanoparticles accumulated at the test line of an LFA, concentrations that may not be possible, or would be difficult, to visualize through traditional optical or fluorescent readers. This in turn correlates to a significant increase in the sensitivity of the LFA, which could open the door to the diagnosis, prognosis, and/or monitoring of infectious diseases or biomarkers, including those that exhibit very low physiological concentrations currently undetectable by optical or fluorescent LFAs. At least some implementations described herein improve over alternative existing approaches for using magnetic lateral flow assays to measure the accumulation of magnetic nanoparticles at the test and control lines, for example: giant magnetoresistance (GMR), bridge circuits (e.g. Wheatstone bridge) or resonance magnetometry. Such existing approaches, which utilize benchtop readers specific to the LFA, are bulky, expensive, and/or not suited for diagnosis and monitoring at the point-of-need (PON, i.e. at-home). Moreover, rather than use a system of cameras, lenses, diodes, and filters to detect the presence of nanoparticles at the test and control line, at least some implementations described herein use LFAs where the backing card (traditionally the physical support for the membranes that make up the LFA) thereof may be replaced by a PCB with sensors that are fixed in position relative to specific positions of the LFA using an adhesive substrate, as described herein. Alternatively or additionally, the PCB is added in addition to the backing card, for example the PCB is connected to a backed LFA (i.e., an LFA comprising a backing card) via an adhesive substrate such that the backing card is sandwiched between the PCB and membrane components of the LFA.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

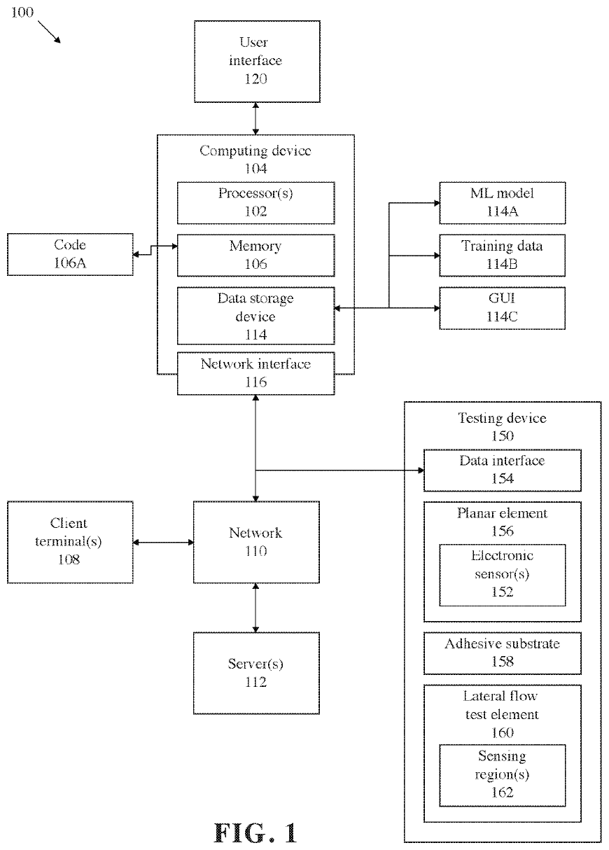

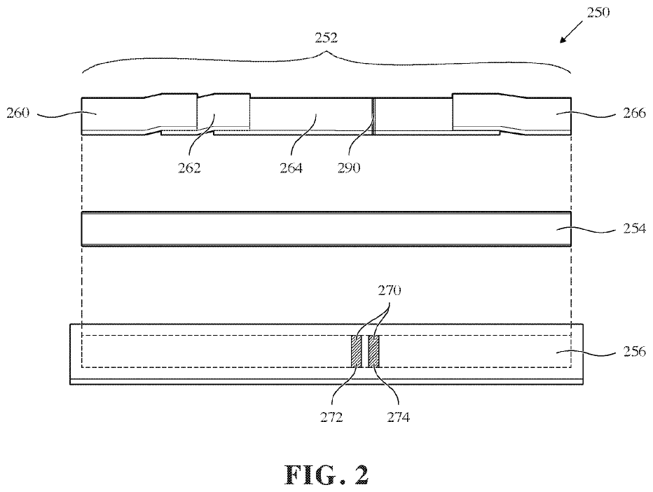

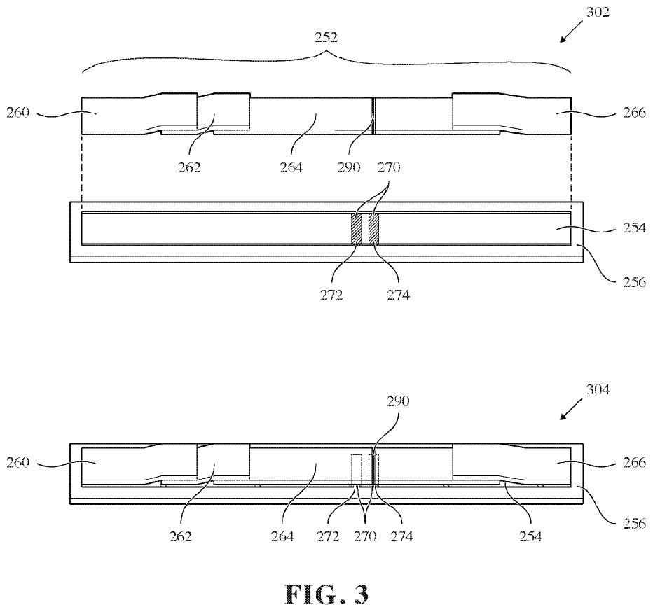

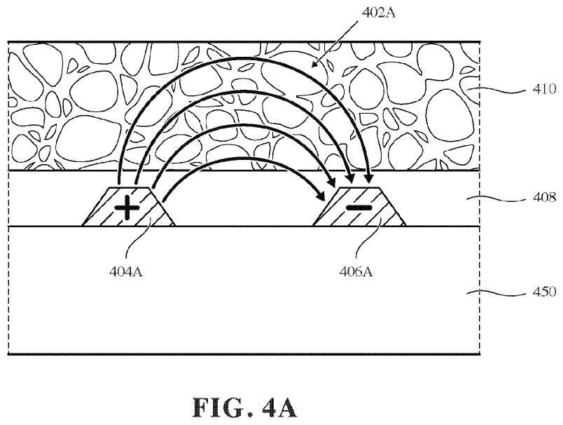

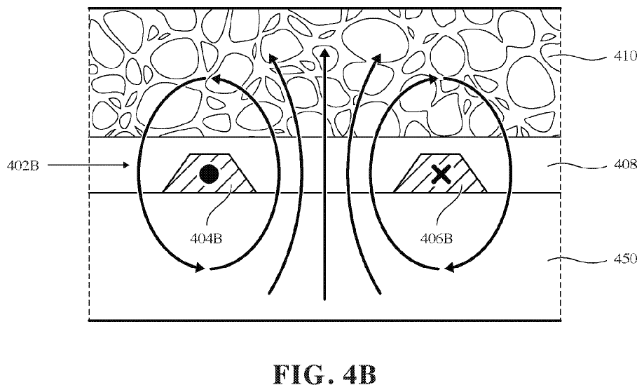

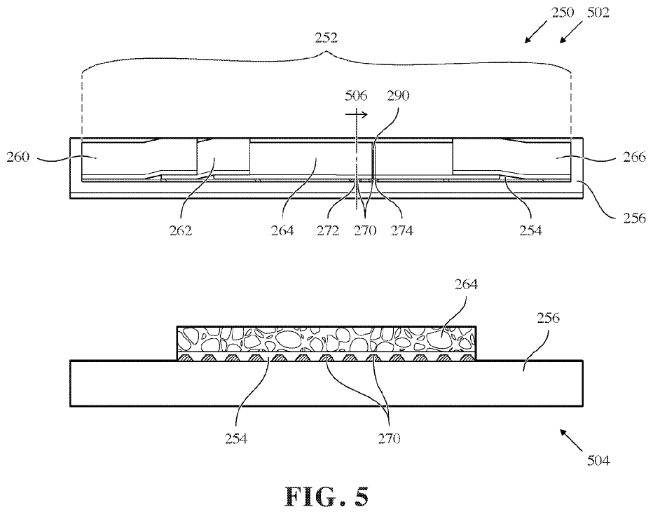

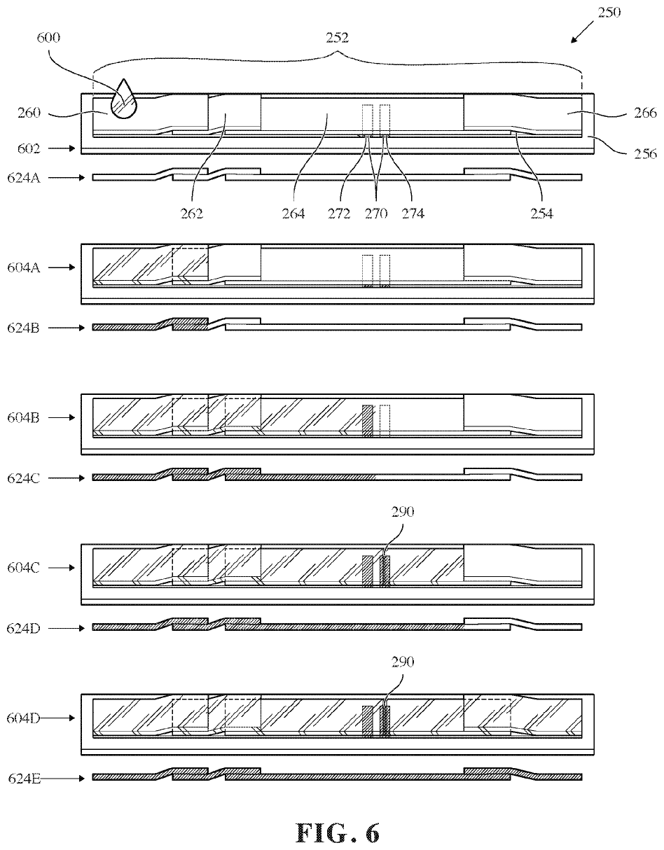

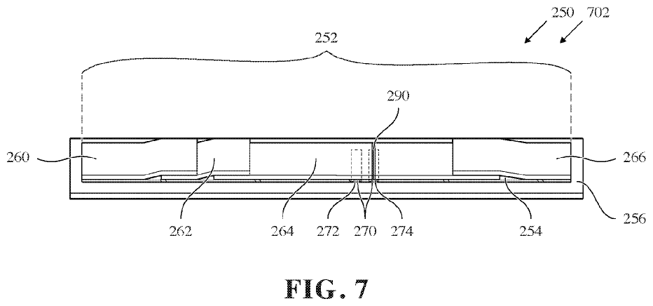

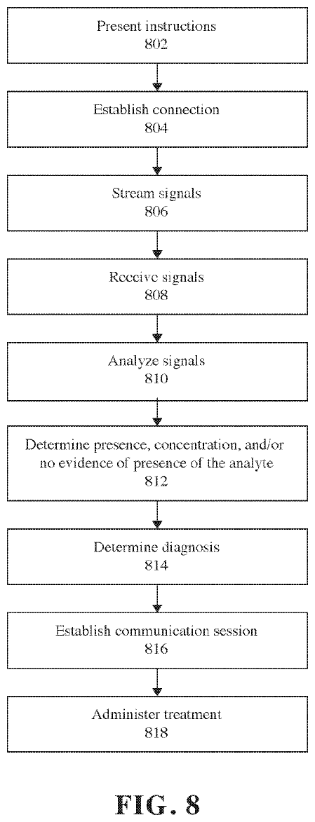

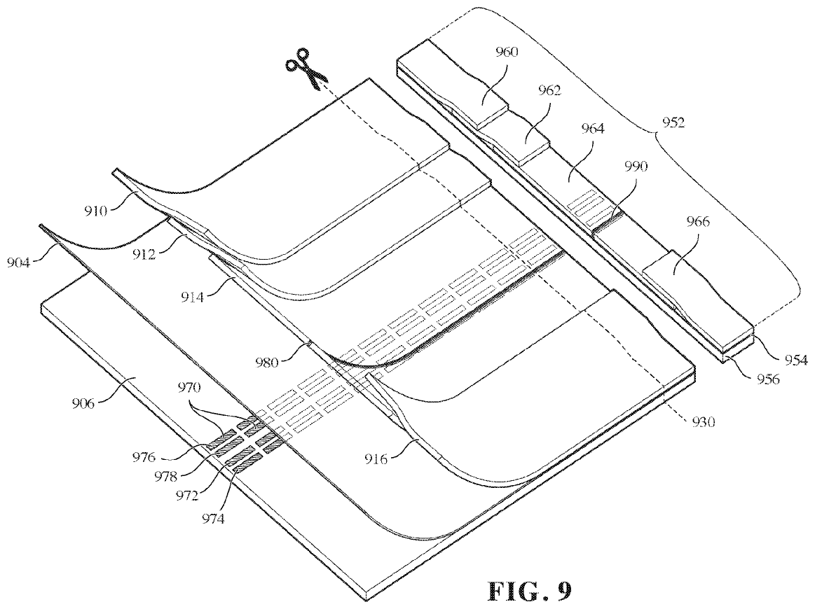

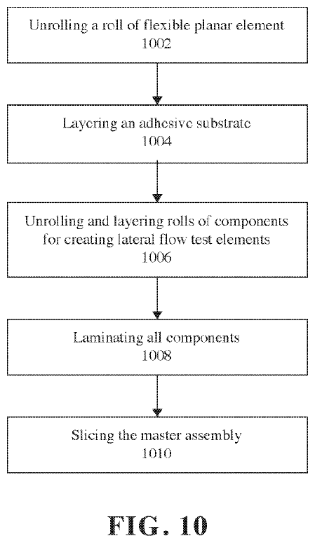

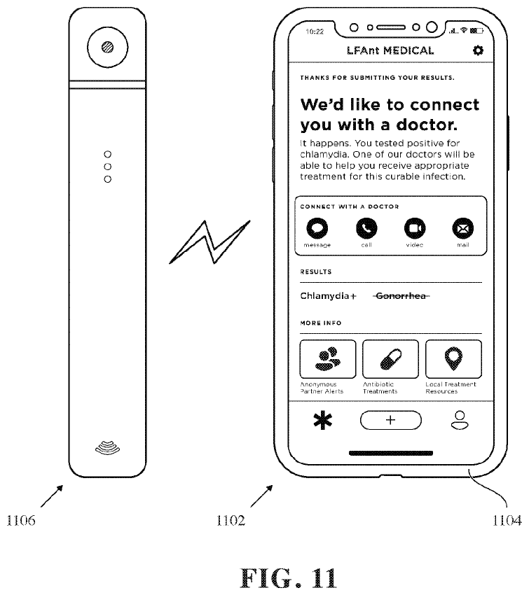

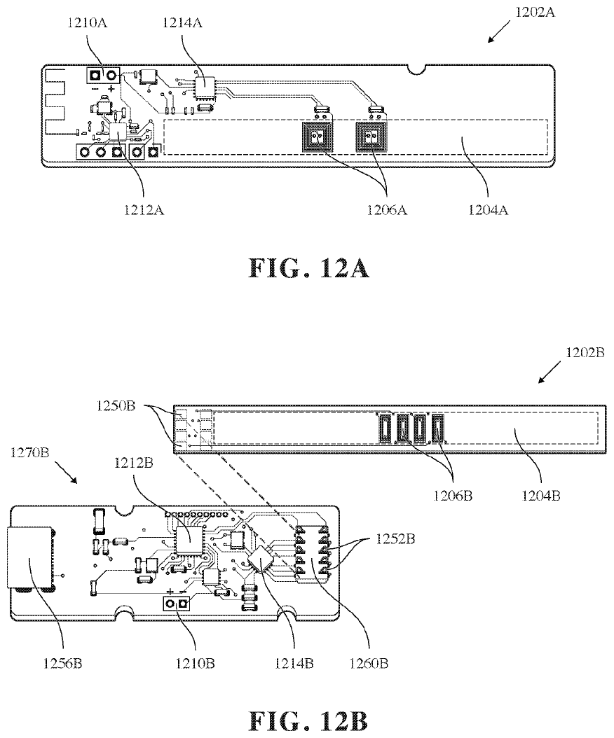

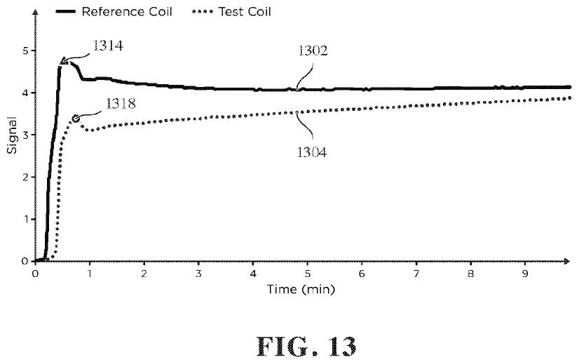

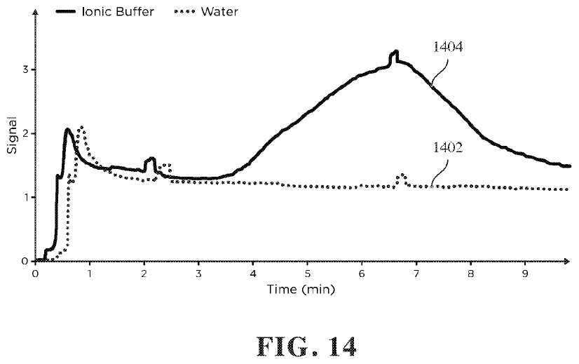

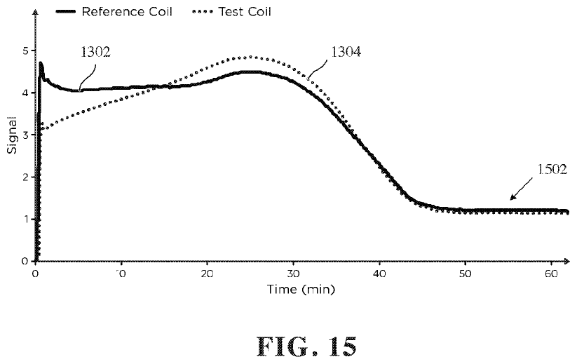

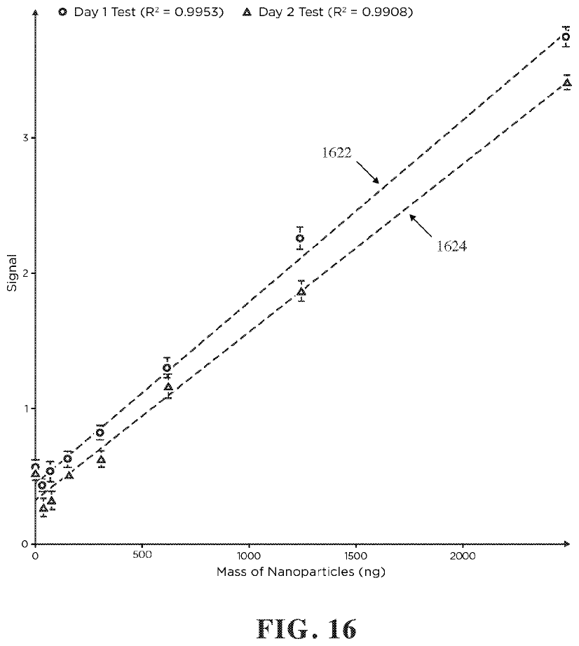

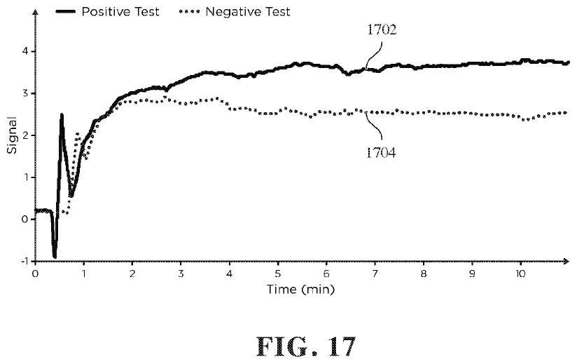

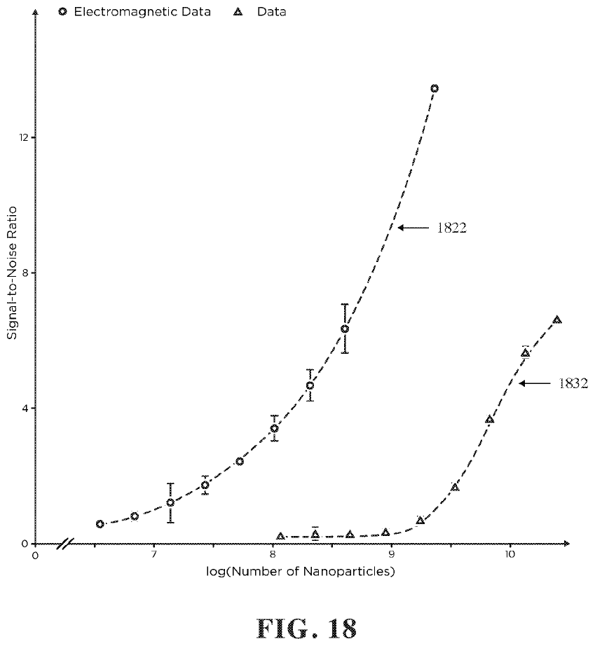

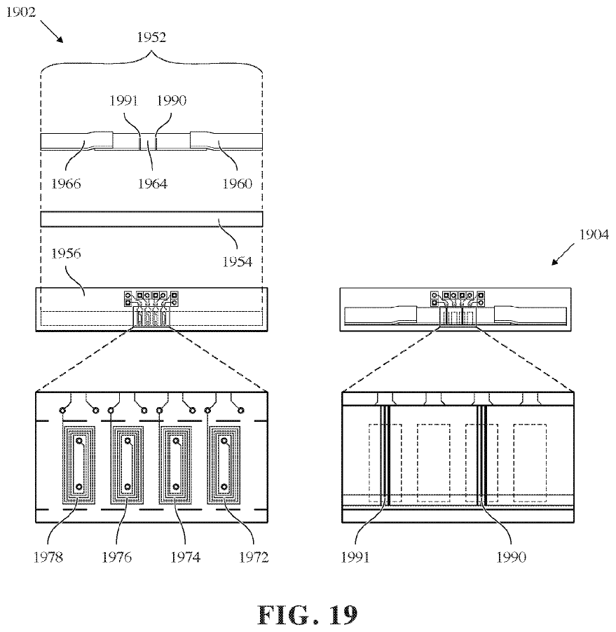

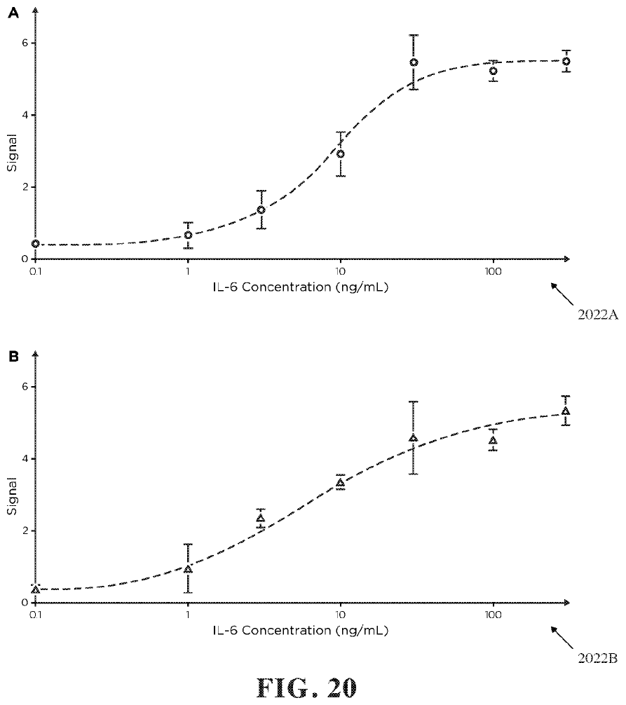

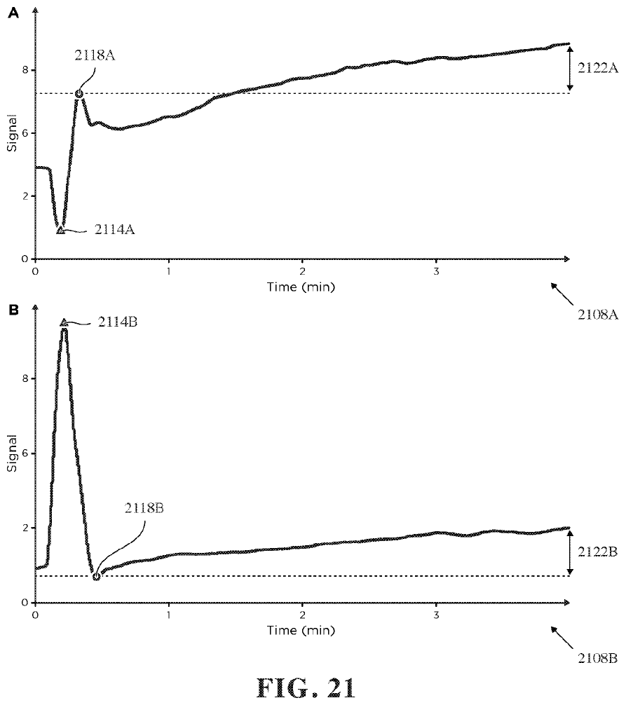

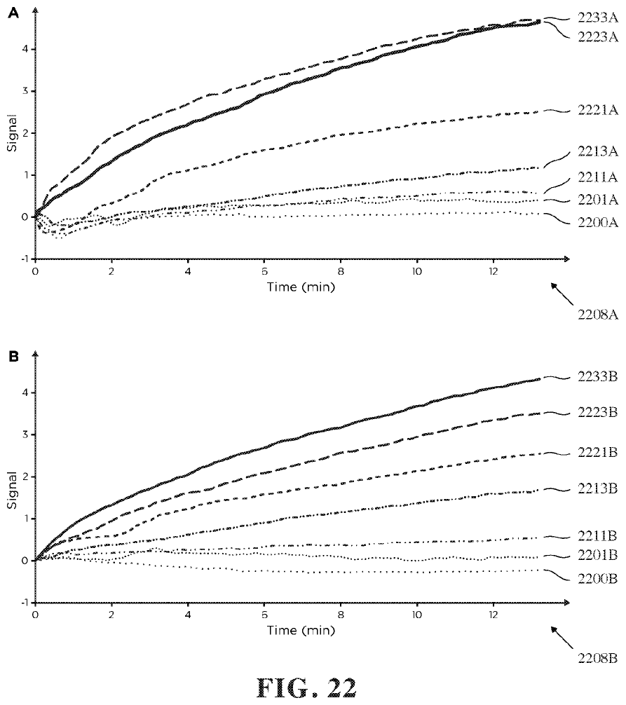

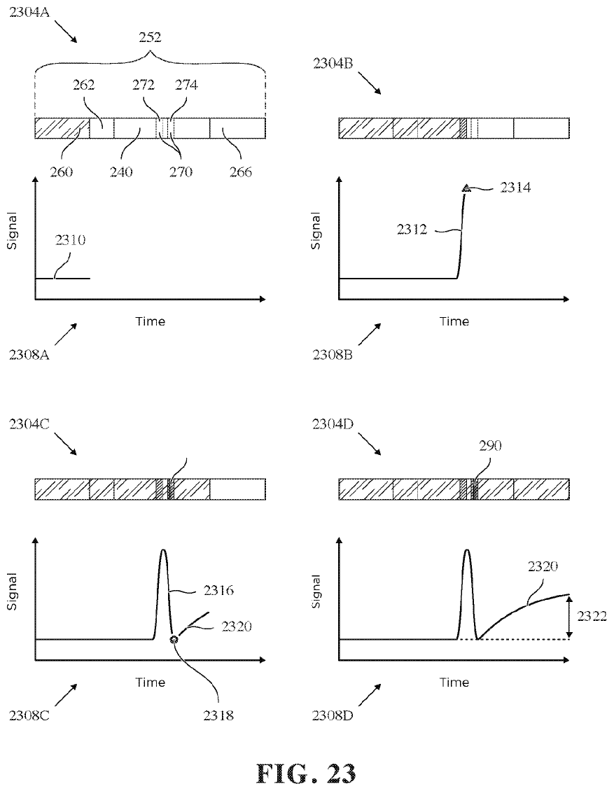

Reference is now made to FIG. 1, which is a block diagram of components of a system 100 for evaluating an analyte, in accordance with some embodiments of the present invention. Reference is also made to FIG. 2, which is a schematic of components for assembling an exemplary testing device 250 for evaluation of the analyte, in accordance with some embodiments of the present invention. It is noted that other components described with reference to FIG. 1 are designed to be used with testing device 250 for testing for the analyte, for processing signals, for example, signal acquisition circuit, battery, wireless (e.g., Bluetooth) data interface, and the like. Reference is also made to FIG. 3, which depicts the process of assembling testing device 250, from components 252, 254, and 256, as described with reference to FIG. 2., in accordance with some embodiments of the present invention. Reference is also made to FIGS. 4A-B, which are schematics of electric field lines between sensors of the planar element (e.g., PCB) of the testing device, in accordance with some embodiments of the present invention. Reference is also made to FIG. 5, which is a schematic of cross section of an assembled testing device 250, shown in a perspective view 502 and cross-sectional view 504 in accordance with some embodiments of the present invention. Reference is also made to FIG. 6, which is a schematic depicting application of a sample to testing device 250, in accordance with some embodiments described herein. Reference is also made to FIG. 7, which is a schematic 702 depicted as a state of diagnostic testing device 250 at the end of its lifecycle. i.e., when the liquid has travelled through the multiple membrane components of LFA 252 and into wick 266, such as following schematic 604D of FIG. 6, in accordance with some embodiments of the present invention. Reference is also made to FIG. 8, which is a schematic of an exemplary process for analyzing signals obtained from a testing device, in accordance with some embodiments of the present invention. Reference is also made to FIG. 9, which is a schematic depicting a perspective view of a small section of the lamination process typical of roll-to-roll manufacturing of testing devices that include LFA connected to a PCB via an adhesive substrate, in accordance with some embodiments of the present invention. Reference is also made to FIG. 10, which is a flowchart of an exemplary process of R2R manufacturing of a testing device, in accordance with some embodiments of the present invention. Reference is also made to FIG. 11, which is a schematic depicting an exemplary GUI 1102 presented on a display of a smartphone 1104, presenting instructions for using and/or testing results, of a testing device 1106 that includes a lateral flow assay connected via adhesive tape with PCB that includes sensors, in accordance with some embodiments of the present invention. Reference is also made to FIG. 12A, which is a schematic of an exemplary planar element 1202 implemented as a PCB comprising a resonant magnetometer designed to connect to a LFA via adhesive substrate, in accordance with some embodiments of the present invention. Reference is also made to FIG. 12B, which is a schematic of an exemplary planar element implemented as a PCB designed to connect to a LFA via adhesive substrate and interchangeably connect to an exemplary resonant magnetometer, in accordance with some embodiments of the present invention. Reference is also made to FIG. 13, which is a graph depicting a response curve from a reference coil 1302 and a response curve from a test coil 1304 over time, as measured by inventors, in accordance with some embodiments of the invention. Reference is also made to FIG. 14, which is a graph depicting the phenomenon of ions flowing across coils creating changes to the resonant frequency of the coils and the calculated inductance, as measured by inventors, in accordance with some embodiments of the invention. Reference is also made to FIG. 15, which is a continuation of curves 1302 and 1304 from the graph of FIG. 13, extending for a longer amount of time, in accordance with some embodiments of the present invention. Reference is also made to FIG. 16, which is a graph computed by subtracting inductance of the test coil from the inductance of the reference coil after running the test for long enough that the inductances have stabilized, in accordance with some embodiments of the present invention. Reference is also made to FIG. 17, which is a graph illustrating the difference in the dynamic response between a positive curve 1702 and a negative curve 1704 of the electromagnetic sensing (EMS) modality used in a testing device fabricated by Inventors, in accordance with some embodiments of the present invention. Reference is also made to FIG. 18, which is a graph comparing electromagnetic data 1822 measured using embodiments described herein, to colorimetric data 1832 measured using standard optical approaches, illustrating the advantage electromagnetic sensing (EMS) has over optical sensing methods currently used in the lateral flow industry. Reference is also made to FIG. 19, which is a schematic of an exemplary inductive sensor configuration and immunosensor assembly used in an experiment for testing of magnetic IL-6 lateral flow strips, in accordance with some embodiments of the present invention. Reference is also made to FIG. 20, which includes standard curves presenting the effect of IL-6 concentration on signal readout of two inductive immunosensor compensation schemes used in the experiment that evaluated the testing of magnetic IL-6 lateral flow strips, in accordance with some embodiments of the present invention. Reference is also made to FIG. 21, which includes time series signal profiles of two inductive immunosensor compensation schemes for a test run at 10 ng/ml of IL-6, of the IL-6 experiment, in accordance with some embodiments of the present invention. Reference is also made to FIG. 22, which depicts averaged truncated time series signal profiles of two inductive immunosensor compensation schemes for all of the tests run in the IL-6 experiment, in accordance with some embodiments of the present invention. Reference is also made to FIG. 23, which includes schematics depicting top views of schematics 604A-D described with reference to FIG. 6 and corresponding sensor response curves, in accordance with some embodiments of the present invention.

System 100 may implement the acts of the method described with reference to FIGS. 2-23, by processor(s) 102 of a computing device 104 executing code instructions stored in a memory 106 (also referred to as a program store).

System 100 includes a testing device 150 for testing of analytes, as described herein.

Testing device 150 includes a planar element 156, one or more electronic sensors 152, and an adhesive substrate 158. Testing device 150 is designed for connecting to a lateral flow test element 160 that includes one or more sensing regions 162 that include analyte binding elements designed to bind to the analyte present in a solution applied to lateral flow test element 160.

Testing device 150 may be designed as a single-use device to be disposed of after a liquid sample is applied thereto. Alternatively, testing device 150 may be designed as a disposable cartridge designed to accept a sample and connect to an external reading device. Alternatively, testing device 150 may be implemented as an all-in-one system with built in reading device, which may be disposable.

It is noted that testing device 150 relates to a high level design with different possible implementations. Additional exemplary embodiments are described below, for example, with reference to FIG. 2, FIG. 11, and FIGS. 12A-B.

Adhesive substrate 158 is connected to a surface of planar element 156 and set to connect to a surface of lateral flow test element 160. Adhesive substrate 158 is applied to fix the position and/or orientation of a region(s) of lateral flow test element 160 that includes sensing region(s) 162 to be aligned facing the electronic sensor(s) 152. Sensing region(s) 162 may be aligned and facing electronic sensor(s) 152.

The adhesive substrate is spread over the surface of planar element 156 and the surface of lateral flow test element 160. Adhesive substrate 158 may be spread over at least one electronic sensor(s) 152 and/or a region of lateral flow test element 160 that includes sensing region 162. Alternatively, or additionally, adhesive substrate 158 may be spread externally to electronic sensor(s) 152 and/or the region of lateral flow test element 160 that includes sensing region 162. Adhesive substrate 158 may be sized to substantially match the size of the surface of planar element 156 and/or the size of the surface of lateral flow test element 160, for example, at least about 80%, or 90%, or 100%. The planar element may be at least the size of a nitrocellulose membrane.

The adhesive substrate places sensing region(s) 162 in close proximity to electronic sensor(s) 152, for example, about 5 to 100 micrometers. The proximity of sensor(s) 152 to sensing region(s) 162 enabled by the adhesive substrate improves the output of sensor(s) 152 (e.g., higher SNR, higher sensitivity readings, and other improved measurements as described herein).

The distribution of adhesive substrate 158 may be selected to exclude a significant amount of air from being present between sensing region(s) 162 and electronic sensor(s) 152. It is noted that a negligible amount of air that does not significantly impact transmission of signals through the adhesive substrate may be present. A non-significant amount of air is present in adhesive substrate 158 in continuous contact between electronic sensor(s) 152 and sensing region(s) 162. When adhesive substrate 158 is in contact between sensor(s) 152 and sensing region(s) 162, the absence of air between sensor(s) 152 and sensing region(s) 162, improves the output of sensor(s) 152 (e.g., higher SNR, higher sensitivity readings, and other improved measurements as described herein).

Optionally, adhesive substrate 158 is implemented as a self-adhesive tape. Adhesive substrate 158 has opposing adhesive surfaces. One surface is connected to the surface of planar element 156. The opposite adhesive surface is set to connect to the surface of lateral flow test element 160.

Alternatively or additionally, adhesive substrate 158 comprises glue, tape, and the like. Each one of adhesive substrate 158, planar element 156, and lateral flow test element 160 may be elongated elements that are arranged in parallel to one another, forming a sandwiched architecture. Adhesive substrate 158 is sandwiched between the surface of planar element 156 and the surface of lateral flow test element 160. Adhesive substrate is sandwiched between electronic sensor 152 and the region of the lateral flow test element that includes sensing region 162.

A height (e.g., thickness) of adhesive substrate 158 is selected to be sufficiently thin for obtaining a sufficiently strong signal above a threshold from electronic sensor(s) 152 and sensing region(s) 162 for evaluation of the analyte, while also providing sufficient bonding force to fix planar element 156 in place relative to lateral flow test element 160.

Sensor 152 is positioned on planar element 156 at a location selected for measuring a sufficient signal strength from sensing region 162 of lateral flow test element 160 for evaluation of the analyte.

Optionally, sensing region 162 includes a testing region(s) and a reference region(s) distinct from the testing region. Optionally, at least two sensor(s) 152 are provided, a test sensor(s) for sensing the testing region and a reference sensor(s) for sensing the reference region. The testing region includes analyte binding elements designed to selectively bind to the analyte. The reference region does not include the analyte binding elements. The analyte administered to lateral flow test element 160 flows across the reference region without selectively binding thereto. Test sensor(s) is positioned to sense the testing region(s) that includes analyte binding elements designed to selectively bind to the analyte administered to lateral flow test element 160. Reference sensor is located within planar element 156 and positioned for sensing the reference region. Signals outputted by the test sensor(s) and/or the reference sensor(s) are analyzed to detect evidence of presence of the analyte, the concentration of the analyte, and/or lack of evidence for presence of the analyte, as described herein.

Analyte binding elements of sensing region 162 are designed to selectively bind to and detect the analyte, for example, biomarkers for evaluation of presence of a medical condition in a subject, such as infectious disease biomarkers for evaluation of whether the subject is infected with an infectious disease. Examples of infectious disease biomarkers include markers for sexually transmitted infections (e.g., gonorrhea, chlamydia), and markers for COVID-19 (also known as SARS-CoV-2, and coronavirus). Other examples of analytes to which analyte binding elements of sensor region 162 are designed to bind include health-related biomarkers for evaluation of relevant health conditions, for example, cortisol (e.g., for stress monitoring), melatonin (e.g., for evaluation of sleep), testosterone, interleukon-6 (IL-6) (e.g., for systemic inflammation), progesterone (e.g., for evaluation of fertility), levels of bacteria in food for ensuring safe consumption, tetrahydrocannabinol (THC) (e.g., for screening for drug users), influenza A, influenza B, group A streptococcus (e.g., screening for infectious diseases), procalcitonin. C-reactive protein (e.g., for systemic inflammation), and Escherichia coli (E. coli) (e.g., food safety). The analytes may be, for example, in saliva, urine, blood, and the like, such as salivary cortisol and/or blood-based cortisol.

Sensor 152 may be designed to generate electromagnetic fields with field lines that originate from sensor 152, pass through adhesive substrate 158, pass through sensing region(s) 162, pass back through adhesive substrate 158, and back to sensor 152. The field lines may originate from one part of sensor 152 and terminate at another part of sensor 152. The field lines may originate and terminate at the same part of sensor 152, for example, as described herein with reference to FIGS. 4A-4B. It is noted that the electric field lines in capacitive sensing shown in FIG. 4A are not the same as magnetic field lines in inductive sensing shown in FIG. 4B.

Sensor 152 may be implemented as one or more capacitive sensors for capacitive sensing, and/or as one or more inductive sensors for inductive sensing. The capacitive sensor may include an interdigitated arrangement of one or more pairs of electrodes. Testing device 150 may include circuitry designed to activate the inductive sensor at a resonant frequency thereof.

For implementation using inductive sensing, testing device 150 may include a controller operating at a sampling frequency, for measuring changes in the resonant frequency of sensor 152 indicating binding of analytes to the at least one sensing region. The resonant frequency of sensor 152 may be selected, based on the physical design of sensor152. Exemplary resonant frequencies of sensor 152 may be, for example, in a range of about 1-50 megahertz (MHz), or 0.001-40 MHZ, or 1-10 MHz, or 5-10 MHZ, or other values. A label selected to exhibit superparamagnetic properties may be used to identify the presence of the analyte by the inductive sensor.

The surface of planar element 156 is sized to correspond at least to the surface of lateral flow test element 160. The surface of planar element 156 may be as large, or larger, than the surface of lateral flow test element 160, such that the entire surface of lateral flow test element 160 is connected to planar element 156.

Planar element 156 serves as a physical support of components of the lateral flow test element 160. When standard lateral flow test elements are used in which a backing card is traditionally used, planar element 156 may in some embodiments replace the backing card altogether. In other embodiments planar element 156 may be attached to the backing card via the adhesive substrate 158. Planar element 156 may be implemented as a PCB, where sensor(s) 152 is integrated within the PCB and/or connected to the PCB.

Testing region(s) of the sensing region(s) 162 of lateral flow test element 160 include the analyte binding elements designed to selectively bind to an analyte of interest in the case where there is an analyte present in a sample administered to the lateral flow test element, as described herein. The sensing region is not necessarily the same as the testing region. In some embodiments the sensing region is different from the testing region. In some embodiments the sensing region is the same as the testing region.

Testing device 150 may include a data interface 154 for providing the output of sensors 152 to another computing device 104 for analysis of the signals for detecting presence of the analyte, for example, a transceiver, an antenna, a wireless interface, a wired interface, a virtual interface. The reader component described herein with reference to the two-part system may be implemented by computing device 104. Alternatively, in the single component system, testing device 150 may communicate with computing device 104.

Lateral flow test element 160 may be, for example, a lateral flow assay. In the lateral flow assay implementation, the solution flows through the lateral flow test element by capillary action. In some embodiments, lateral flow test element 160 (also referred to herein as LFA) is designed using the sandwich assay format. This configuration of LFA 160 has analyte binding elements patterned on the testing region, which bind to the analyte present in the sample as it flows through the testing region. Concurrently, labels (e.g., nanoparticles) functionalized with complimentary analyte binding elements also bind to the analyte captured in the test region. Together, these binding events lead to the accumulation of analyte-label complexes in the testing region.

In some embodiments, lateral flow test element 160 is designed using the competitive assay format. This assay format is preferable for small molecule targets such as hormones and vitamins, where steric hindrances make the binding of two biorecognition elements to one analyte unlikely. In this format, LFA 160 has analyte patterned on the testing region before application of the sample. If little or no analyte is present in the sample, the labels will bind to the analyte present in the test region, producing a strong signal. If there is a sufficient concentration of analyte in the sample, most of the labels will bind to the analyte in the sample instead of the analyte patterned on the testing region. Once this has occurred, the label is typically unable to bind to the analyte on the test line, and accordingly as the concentration of analyte in the sample increases, the signal from the testing region will decrease. This is unlike the sandwich assay format, where more analyte in the sample will typically result in a greater testing region signal due to larger amounts of analyte-label accumulation.

It is noted that the design of LFA 160 is not necessarily limited to the aforementioned designs, as a combination of the designs and/or other designs may be implemented.

Computing device 104 obtains data outputted by one or more electronic sensors 152 of testing device 150, optionally via data interface 154. Computing device 104 may analyze the signals outputted by electronic sensors 152 to obtain an outcome, for example, positive or negative for a certain biomarker (e.g., indicative of an infectious disease), diagnosis (e.g., for the infectious disease), and/or amount of the biomarker (e.g., concentration value, and/or within a range).

Computing device 104 may be implemented as, for example one or more and/or combination of: a group of connected devices, a client terminal, a server, a virtual server, a computing cloud, a virtual machine, a desktop computer, a thin client, a network node, and/or a mobile device (e.g., a smartphone, a tablet computer, a laptop computer, a wearable computer, glasses computer, and a watch computer).

Multiple architectures of system 100 based on computing device 104 may be implemented. For example: an architecture designed to provide local services. Computing device 104 may be implemented as a standalone device (e.g., kiosk, client terminal, smartphone) that includes locally stored code instructions 106A that implement one or more of the acts described with reference to FIGS. 2-23. The locally stored instructions may be obtained from another server, for example, by downloading the code over the network, and/or loading the code from a portable storage device. Computing device 104 may obtain signals outputted by sensor 152 of testing device 150, optionally via a network 110, such as a short range wireless connection, and/or cable connection (e.g., USB). Computing device 104 locally analyzes the signals, for example, by feeding the signals into a machine learning model 114A, and/or applying a set of rules such as a threshold. The outcome (e.g., test results and/or diagnosis) may be provided, for presentation on a display 120 of computing device 104 of the specific user, and/or forwarded to a server 112 to enable another user to obtain the results, for example, a server of a healthcare provider and/or public health agency, to obtain assistance from a physician.

In another example, the architecture is designed to provide centralized services. Computing device 104 executing stored code instructions 106A, may be implemented as one or more servers (e.g., network server, web server, a computing cloud, a virtual server) that provides centralized services (e.g., one or more of the acts described with reference to FIGS. 2-23) to one or more client terminals 108 over network 110. Each client terminal 108 is locally connected to a respective testing device 150, via network 110 and/or another connection, such as a short range wireless connection, cable (e.g., USB), and the like. Computing device 104 may provide, for example, software as a service (SaaS) to client terminal(s) 108, software services accessible using a software interface (e.g., application programming interface (API), software development kit (SDK)), an application for local download to client terminal(s) 108, an add-on to a web browser running on client terminal(s) 108, and/or functions using a remote access session to client terminals 108, such as through a web browser executed by client terminal 108 accessing a web sited hosted by computing device 104. Signals obtained by each respective client terminal 108 from respective sensors 152 of respective testing devices 150 are provided to computing device 104 via one or more of the approaches described previously. Computing device centrally analyzes the respective signals, for example, by feeding the respective signals into machine learning model 114A and/or applying a set of rules (e.g., threshold), and provides the outcome (e.g., diagnosis) to respective client terminals 108 (e.g., for presentation on a display) and/or to server(s) 112 such as of healthcare providers and/or public health authorities.

It is noted that ML model(s) 114A may be trained on training data 114B, as described herein. The training may be performed by computing device 104 and/or by another device that provides trained ML model(s) 114A to computing device 104.

Hardware processor(s) 102 of computing device 104 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include a single processor, or multiple processors (homogenous or heterogeneous) arranged for parallel processing, as clusters and/or as one or more multi core processing devices.

Memory 106 stores code instructions executable by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Memory 106 stores code 106A that implements one or more features and/or acts of the method described with reference to FIGS. 2-23 when executed by hardware processor(s) 102. Memory 106 may store other data, for example, test identification (ID) associated with batch-specific calibration data.

Computing device 104 may include a data storage device 114 for storing data, for example, the machine learning model(s) 114A and/or other code for analysis of the signals (e.g., set of rules, threshold) described herein, and/or a training data repository 114B that includes data for training of ML model(s) 114A, and/or GUI code 114C for providing an interactive display for presenting the results of the test on a user's client terminal and/or for establishing a communication session with a remote entity such as a healthcare provider. Data storage device 114 may be implemented as, for example, a memory, a local hard-drive, virtual storage, a removable storage unit, an optical disk, a storage device, and/or as a remote server and/or computing cloud (e.g., accessed using a network connection).

Network 110 may be implemented as, for example, the internet, a local area network, a virtual network, a wireless network, a cellular network, a local bus, a point to point link (e.g., wired), and/or combinations of the aforementioned.

Computing device 104 may include a network interface 116 for connecting to network 110. Network interface 116 of computing device 104 and/or data interface 154 of testing device 150 may be implemented, for example, as one or more of, a network interface card, a wireless interface to connect to a wireless network, a physical interface for connecting to a cable for network connectivity, a virtual interface implemented in software, network communication software providing higher layers of network connectivity, and/or other implementations. It should be noted that network interface 116 may connect to data interface 154 of testing device 150 without necessarily going through network 110 and/or via another connection, for example, a short range wireless connection and/or a cable.

Computing device 104 may connect using network 110 (or another communication channel, such as through a direct link (e.g., cable, wireless) and/or indirect link (e.g., via an intermediary computing unit such as a server, and/or via a storage device) with one or more of:
Remote server(s) 112, for example, of a healthcare provider and/or public health office. Communication sessions may be established between client terminal 108 and/or computing device 104 and/or server(s) 112, for example, to provide telemedicine consultations regarding the results.
Client terminal(s) 108, when computing device 104 is implemented as a server remotely providing the features and/or acts described herein.
Data interface 154 of testing device 150 to obtain data outputted by sensor(s) 152.

Computing device 104 and/or client terminal(s) 108 include and/or are in communication with one or more physical user interfaces 120 that include a mechanism for a user to enter data (e.g., request telemedicine consult) and/or view the displayed results (e.g., diagnosis), within a GUI (e.g., generated based on GUI code 114C). Exemplary user interfaces 120 include, for example, one or more of, a touchscreen, a display, gesture activation devices, a keyboard, a mouse, and voice activated software using speakers and a microphone.

Referring now back to FIG. 8, at 802, prior to beginning the assay, instructions may be presented, for example, on a display of a mobile device and/or other computing device. The instruction may request that the user connect their mobile device (e.g., smartphone) to the testing device, for example, through an in-app prompt.

At 804, a connection between the mobile device and the testing device may be established, for example, using a short range wireless connection (e.g., Bluetooth low energy), and/or other close-range connection technologies. The testing device may be powered, for example, by wireless charging via interaction with the user's mobile device, and/or by a small battery.

At 806, once the connection is established, the assay readings are collected on the testing device, where the readings may be continuously streamed (and/or intermittently transmitted) to the nearby connected smart device via a wireless protocol such as Bluetooth low energy. In some embodiments, the communications electronics are embedded on the testing device directly adjacent to the sensors. In other embodiments, the communications electronics and/or other circuitry are separate from the testing device.

The data is not necessarily stored on-device. While a memory chip may be included, the data may be streamed in real-time from the onset of the test. The data may thus be structured as a multidimensional time-series, where each test signal includes thousands of test and reference sensor sample readings, taken over the duration of the test.

At 808, signals are obtained from the sensor(s) of the testing device, as described herein. The signals may be obtained by the computing device (e.g., smartphone), optionally via a connection, such as a network connection over a network, for example, a short range temporarily established wireless connection.

Signals may be obtained after a liquid has been applied to the testing device, as liquid is being applied, and/or prior to liquid being applied, and the liquid has flowed over the sensing region facing the sensor (e.g., drawn by capillary action), as described herein.

Signals may be obtained from capacitive sensor(s) for capacitive sensing, and/or inductive sensor(s) for inductive sensing.

Signals are obtained from the sensor sensing the sensing region of the lateral flow test element. The sensing region includes analyte binding elements designed to bind to the analyte administered to the lateral flow test element. The signals may indicate a concentration of the analyte and/or evidence of presence of the analyte or lack of evidence indicating presence of the analyte binding to the analyte binding elements.

Signals may further be computed and/or obtained from signals outputted by a test electronic sensor that senses the testing region and a reference electronic sensor that senses the reference region. The reference region is distinct and apart from the sensing region. The analyte which selectively binds to the analyte binding elements in the testing region does not selectively bind to the reference region. Analyte binding elements are absent from the reference region.

When sufficient concentration of the analyte is present in the sample, obtained signals (e.g., generated by the nanoparticles) are expected to be of sufficient signal strength (e.g., above a threshold) for detecting presence of the analyte, based on the proximity of the sensor to the sensing region obtained by the adhesive substrate. The adhesive substrate provides the proximity by being connected to the surface of the planar element and to the surface of the lateral flow test element for fixing the electronic sensor in a position facing a region of the lateral flow test element that includes the sensing region, as described herein.

At 810, the signals are analyzed for measuring accumulation of the analyte at the sensing region of the lateral flow test element. The analysis of the signals may be performed locally by the computing device (e.g., smartphone) and/or remotely by a server in communication with the computing device (e.g., signals are transmitted to the server via the computing device).

As the assay is performed and associated data streamed to the accompanying reading device (e.g., smartphone, external reader, or part of the all-in-one architecture), the mobile device begins processing the data to ascertain the presence of an analyte.

As used herein, the term “reader” or “reading device” refers to the reader component which may be built into in the all-in-one system (i.e., the testing device), or included as a part of the multi-part system.

There are one or more exemplary approaches for analyzing the data:
The value of the readings relative to a standard curve is taken.
Analytical transformations and filters are performed on the signal to improve the diagnostic accuracy. These include, but are not limited to, frequency space transformations, curve fitting, direct analytical analyses, etc.
The smart device application may store a machine learning model trained to classify signals based on the testing scenario. This machine learning model may be trained on the time-series data collected from labelled samples with known concentrations of the analyte. This training may occur in a laboratory setting and results in parametric weighting of the machine learning (ML) model's architecture prior to it being stored on the smart device. Notably, this ML model is created such that the user's test processing may be done entirely on-device, with no communication with a remote server. The ML model may employ a custom framework to achieve positive results on each specific testing scenario but is based on state-of-the-art time series analysis processes developed and open-sourced by multiple contributors in the AI community.

The signals may be analyzed by feeding the signals into the ML model, for example, a classifier, a statistical classifier, one or more neural networks of various architectures (e.g., convolutional, fully connected, deep, encoder-decoder, recurrent, graph, combination of multiple architectures), support vector machines (SVM), logistic regression, k-nearest neighbor, decision trees, boosting, random forest, a regressor and the like. The ML model may be trained using supervised approaches and/or unsupervised approaches on a training dataset. The ML model may be trained on a training dataset that includes records, each record including a sample signal labelled with a ground truth indication of at least one of: presence of the analyte, lack of evidence indicating presence of the analyte (e.g., absence of the analyte, lack of sufficient concentration of the analyte), and concentration of the analyte.

The signals may be analyzed using other approaches, for example, a set of rules, a mathematical equation, and/or other deterministic approaches.

At 812, the presence of the analyte and/or concentration of the analyte is determined according to the analysis. The lack of presence of the analyte may be determined according to the analysis. Lack of presence of the analyte may be due to absence of any analyte, or lack of sufficient detectable concentration of the analyte.

At 814, a diagnosis of a medical condition of the subject is determined according to the evidence indicating presence of the analyte, the concentration of the analyte, and/or lack of evidence indicating presence of the analyte.

Diagnosis may be made, for example, when the analyte is present, when the analyte is undetected, when the concentration of the analyte is above a threshold, and when the concentration of the analyte is below the threshold. For example, lack of detection of the analyte may be used to exclude medical conditions, for example, confirm that the user is not infected by an infectious agent.

The medical condition may be, for example, infection with an infectious disease agent, such as COVID-19, or a STI such as chlamydia, or gonorrhea.

Optionally, once the test has been performed, there may be two possible outcomes. In the first, the test is invalid. An invalid test may arise from failure modes, for example, the use of an expired testing device, the re-use of a previously used testing device, and/or technical issues with components of the testing device (e.g., sensors, integrated circuits, sample fluid flow through the test strip). In this case, an automated attempt at understanding the error will be made, and the issue conveyed directly to the user in-app. A recommendation to pursue additional testing will be made. Secondly, in the case of a valid test, the user will be provided with their result through the application's interface on their smart device. In some embodiments, this could be in the form of a positive versus negative diagnosis for a single or many infection(s), and in others, the detected concentrations of important biomarkers. In the case of illnesses or infections that are dictated by the concentration and not the presence of an analyte, the user will be presented with the associated information.

At 816, in response to the valid test result, a communication session with a remote client terminal of a healthcare provider and/or public health entity may be established. For example, a video call, a chat session, a voice call, and the like.

Alternatively or additionally, when a user is provided with a valid test result corresponding to a diagnosis for a health condition, the application may utilize the location services or user-entered data to ascertain the region in which the test is being performed. Given this information, the application may then determine whether compliance with regional health authorities is necessary. The application may report the condition while anonymizing as much information as allowed under the jurisdiction, as an example, for reportable diseases. Simultaneous to the diagnosis, the user may also be given access to professionally verified learning materials as well as telemedicine services. Given the proper regional background, the telemedicine services may be communicated the test results via API integration to provide appropriate care for the user, including but not limited to remote care and prescription of medication.

At 818, a treatment that is effective for treating the medical condition may be prescribed to user (e.g., patient), for example, by the healthcare provider in response to the communication session, and/or automatically generated in response to the diagnosis.

The treatment may be, for example, a prescription for antibiotics for treating a sexually transmitted infection.

Attention is now directed back to FIG. 2, which is a schematic of components for assembling an exemplary testing device 250 for evaluation of an analyte, in accordance with some embodiments of the present invention. Testing device 250 is assembled from a lateral flow assay 252 (also referred to herein as lateral flow test element), an adhesive substrate 254, and a planar element 256, optionally elongated. Testing device 250 may be implemented based on testing device 150 described with reference to FIG. 1.

LFA 252 may include the following components, which may be standard: sample pad 260, conjugate pad 262, test pad (e.g., nitrocellulose (NC) membrane, synthetic paper) 264, and wick 266. Standard LFAs are physically supported by a semi-rigid plastic structure called a backing card. LFA 252 may be a standard LFA without the standard backing card (e.g., backing card removed, backing card not assembled), also referred to herein as unbacked LFA.

Adhesive substrate 252 may be tens of microns thin, for example, KN2211 adhesive from Kenosha Tapes, and in other implementations be thinner than 10 μm, for example, ultra-thin PET-based 5 μm double sided tape (No. 5600) from Nitto Denko.

Planar element 256 may be implemented as a printed circuit board (PCB). PCB 256 may replace the standard backing card, providing physical support for LFA 252. PCB 256 includes sensors 270, which may be capacitive sensors and/or inductive sensors. Sensors 270 may include a test sensor 274 and a reference sensor 272 for example, as described herein.

Adhesive substrate 252 that binds PCB 256 to LFA 252 enables accurate collection of sensor measurements (e.g., capacitive and/or inductive data) at specific preselected locations of LFA 252, optionally in real time.

Attention is now directed back to FIG. 3, which depicts the assembly of testing device 250, from components 252, 254, and 256, as described with reference to FIG. 2., in accordance with some embodiments of the present invention. Schematic 302 depicts adhesive substrate 254 connected to PCB 256, and unbacked LFA 252 which is not yet connected to adhesive substrate 254. LFA 252 is lined up with respect to adhesive substrate 254, which is connected to PCB 256, for positioning LFA 252 with respect to sensors within PCB 256. In particular, test line 290 of LFA 252 is fixed in position over and/or in close proximity to sensor(s) 270 of PCB 256 by adhesive substrate 254, as described herein. Schematic 304 depicts a fully assembled testing device 205, assembled from components 252, 254, and 256, as described herein.

Attention is now directed back to FIGS. 4A-B, which are schematics of electromagnetic field lines between sensors of the planar element (e.g., PCB) of the testing device, in accordance with some embodiments of the present invention. FIGS. 4A-B illustrate the significance of the adhesive substrate fixing the position of the sensors of the PCB relative to specific regions of the unbacked LFA, as described herein.

FIG. 4A depicts electric field lines 402A passing between electrodes 404A and 406A implemented as a planar capacitor. It is noted that although one capacitor is shown, it is to be understood that two or more capacitors may be implemented. Sensors 404A and 406A are located on planar element 450 of the testing device. Planar element 450 may be implemented as a PCB board, as described herein. Electrodes 404A and 406A, such as planar capacitors, are arranged along a plane. Such planar arrangement of electrodes 404A and 406A is unlike in a parallel plate capacitor setup where electrodes are placed on either side of the material whose capacitance is being probed. Field lines 402A leave the plane in which electrodes 404A and 406A reside, travelling upwards in an arc-like path, passing through adhesive film 408 and nitrocellulose membrane 410 (or other implementation for the test pad) of the testing device, probing the capacitance of adhesive film 408 and nitrocellulose membrane 410, before returning to the other electrode 404A or 406A.

FIG. 4B depicts magnetic field lines 402B generated by coil traces (referred to as traces) 404B and 406B implemented as planar inductive sensors. Traces 404B and 406B are located on planar element 450 of the testing device. Planar element 450 may be implemented as a PCB board, as described herein. Field lines 402B pass through planar element 450, adhesive film 408, and nitrocellulose membrane 410 of the testing device, probing the inductance of planar element 450, adhesive film 408, and nitrocellulose membrane 410.

Attention is now directed back to FIG. 5, which shows a schematic of an assembled testing device 250, shown in a perspective view 502 and cross-sectional view 504 of cross-section 506, in accordance with some embodiments of the present invention. FIG. 5 illustrates the significance of adhesive substrate 254 fixing the position of the sensors of the PCB relative to specific regions of the unbacked LFA, as described herein. Testing device 250 and components thereof are as described herein, such as FIG. 2-3.

The signal produced by an LFA may be optically resolved as the appearance of a visible line over the lifetime of the test, which is caused by the accumulation of nanoparticle labels, for example, at test line 290. In the embodiment of capacitive sensors described herein, the nanoparticle labels may be metallic (e.g. gold, silver, iron, etc.). Inductive sensors may use magnetic nanoparticle labels (e.g. iron oxide, magnetite, etc.). Achieving alignment between test line 290 and sensors (e.g., the centerline of the long axis of the sensor is aligned and facing the centerline of the long axis of test line 290), which is obtained and fixed by adhesive substrate 254, maximizes the interaction between the electromagnetic field lines of the sensors and the volume within which the nanoparticles will be immobilized in the context of a visual signal output, denoted by test line 290, as described herein, such with reference to FIGS. 4A-B.

LFA 252 is designed to enable a sample fluid containing an analyte of interest to flow through a series of overlapping membranes which have been treated with specific chemical and biological reagents, i.e., components 260, 262, 264, and 266. These reagents allow for the travel of the sample and analyte as well as the capture of the analyte at specific physical locations along the test pad 264. The location at which the signal may be visually interpreted based on the accumulation of nanoparticle labels may be depicted by the development of an optical readout (e.g., colorimetric or fluorescent) at the test line 290, which may appear over time. Test line 290 is set to be in alignment with test sensor 274. To the left of the test sensor 274 is a reference (or ref) sensor 272, which is positioned close to the test sensor 274 and is used as a negative control against which the response of the test sensor 274 over the lifetime of the assay can be compared to determine positive or negative results based on nanoparticle accumulation.

Attention is now directed back to FIG. 6, which is a schematic depicting application of a sample to testing device 250, in accordance with some embodiments described herein.

Schematic 602 depicts application of a biological fluid sample 600 to testing device 250. Biological fluid sample 600 presumably contains the analyte of interest for the specific instance of the diagnostic testing device 250. Fluid sample 600 is added to sample pad 260. The sample matrix as well as the sample volume will differ depending on the application.

Schematics 604A-D depicts the flow of sample 600 through the LFA 252. The porous membranes of the LFA, when wetted, allow for liquid movement through capillary action. Flow lines 624A-E provides a cross sectional indication of how far along testing device 260 fluid sample 600 has travelled with respect to schematics 604A-D.

Schematic 604A depicts fluid sample 600 traveling from sample pad 260 to conjugate pad 262, where nanoparticles conjugated to detection molecules await.

Schematic 604B depicts fluid wetting the test pad 264 above both sensors 270 (e.g., capacitors and/or inductors 272, 274). Fluid travels faster than the analyte-label complexes through the porous membrane, and the areas above the reference 272 and test 274 sensors become saturated with sample fluid.

Schematic 604C depicts analyte-label complexes beginning to accumulate at the test line 290 above the test sensor 274. Analyte binding elements patterned on the test pad 264 above the test sensor 274 cause the accumulation of the complexes whereas such complexes do not selectively bind above the reference sensor 272.

Schematic 604D depicts sample fluid being absorbed by wick 266. Highly absorbent material comprising the wick 266 creates a fluid sink, allowing the movement of the sample volume across the sensors 270.

Attention is now directed back to FIG. 7, which is a schematic 702 depicted as a state of diagnostic testing device 250 at the end of its lifecycle, i.e., when the liquid has travelled through the multiple membrane components of LFA 252, such as following schematic 604D of FIG. 6, in accordance with some embodiments of the present invention. The accumulation of nanoparticles at the capture region above the test sensor 274 has resulted in an optically resolvable signal consisting of an optical readout (e.g., colorimetric or fluorescent) at test line 290. Thus, the volume of the test pad 264 above test sensor 274 gradually develops different dielectric properties due to the accumulation of the nanoparticles when compared to the rest of the test pad 264, including the volume directly over the reference sensor 272. This accumulation process, monitored by periodically probing the capacitance in the case of capacitive sensors, or inductance in the case of inductive sensors, of the material at specific selected frequencies (for example, about 1-50 Hertz (Hz)) over the entire lifetime of the test enables distinguishing differences in the time-series capacitance/inductance curves generated at the test 274 and reference 272 sensors. These differences enable distinguishing between positive and negative results electronically and without needing to rely on the visual signal output alone or at all. For example, in the case of an assay performed by a user with extremely high concentrations of the analyte in its applicable biological matrix, the accumulation of analyte-label complexes at the test line 290 might be significant enough that a positive result can be ascertained before the appearance of any resolvable visual signal. Exemplary potential advantages of embodiments and/or implementations described herein include: greater sensitivity, faster time to results, improving usability of the device, and/or automating results interpretation.

Attention is now directed back to FIG. 11, which is a schematic depicting an exemplary GUI 1102 presented on a display of a smartphone 1104, presenting instructions for using and/or testing results, of a testing device 1106 that includes a lateral flow assay connected via adhesive tape with PCB that includes sensors, in accordance with some embodiments of the present invention. GUI 1102 may present instructions, test results, and/or option to establish a communication session with a healthcare provider, for example, as described with reference to FIG. 8.

Attention is now directed back to FIG. 9, which is a schematic depicting a perspective view of roll-to-roll manufacturing of testing devices that include LFA connected to a PCB via an adhesive substrate, in accordance with some embodiments of the present invention.

A reel of backing structure 906 (e.g., PCB), comprising sensors 970, is layered with a reel of self-adhesive 904 (also referred to herein as adhesive tape), and sequentially laminated with reels of sample membrane 910, conjugate membrane 912, test membrane 914, and wicking membrane 916 to form a master assembly, in an order conducive to proper overlapping for the function of the LFA.

Alternatively, or additionally, in other embodiments, reels of cover tapes as well as other LFA components may be laminated onto the reel of backing structure 906, and the reels of membrane-based components 910, 912, 914, and 916 may be modified and/or removed depending on the LFA-based biosensor of interest.

A section of the test membrane 914 is patterned with a capture line 980, which is immobilized along the length of the test membrane 914. The capture line 980 includes analyte binding elements, for example, antibodies, aptamers, and/or enzymes, which are selected based on their affinity to at least one specific analyte of interest. The capture line 980 is not necessarily visible to the naked eye before application of a fluid sample but is shown for reference. The capture line 980 may be made visible to ensure proper alignment over the sensors using dyes such as Orange G, which may dissolve as fluid is running through the strips and should not affect the functionality of the analyte binding elements or optical characteristics of the test.

The reels of membrane-based components 910, 912, 914, and 916 are continuously laminated onto the reel of backing structure 906 at one end of the assembly. Thereafter, individual lateral flow strips 952, which represent individual diagnostic testing devices, are cut from the master assembly at evenly spaced intervals 930. Laminating may be done by sandwiching the self-adhesive tape between the planar element and the components of the testing device. The testing devices may be cut from the same end as where the lamination ends. The master assembly may be transferred from the lamination equipment to a reel-fed guillotine for cutting. Lateral flow strips 952 can be cut from the master assembly using an automated guillotine to improve accuracy and reproducibility during the strip cutting process.

The lateral flow strip 952 comprises a backing structure 956, a self-adhesive layer 954, a sample pad 960, a conjugate pad 962, a test pad 964, a wicking pad 966, and a test line 990 (line 990 does not have to be a test line, but could be a control line or other type of line for providing quality control), which share the same compositions as the reel of backing structure 906, the reel of self-adhesive 904, the reels of sample membrane 910, conjugate membrane 912, test membrane 914, wicking membrane 916, and capture line 980, respectively.

The reel of backing structure 906, may comprise a flexible PCB, commonly composed of polyimide. The sensors 970 may be etched onto the reel of backing structure 906 and composed of metallic traces generally made of copper. The reel of self-adhesive 904 is composed of an adhesive which is typically less than 100 microns thick and generally comprises a polyester layer sandwiched between two layers of acrylic adhesive.

There may be two pairs of sensors 970 on each lateral flow strip in the present embodiment: a sensor pair of a test sensing region comprises sensors 972 and 974, and a sensor pair of a compensation or reference sensing region comprises sensors 976 and 978. Each pair of sensors 970 may include a test sensor and a reference sensor. Test sensor 974 in the sensor pair of the test sensing region may be aligned and optionally centered over the test line 990 on the lateral flow strip 952. The alignment may be that the test sensor 974 is facing the test line 990.

Test sensor 974 and reference sensor 972 of the test sensing region are used to measure changes in the local properties of the lateral flow strip 952 at the test line 970 due to an accumulation of captured analyte-label complexes. In the case that the sensors 970 (i.e., each sensor) comprises one or more planar coils with at least one layer designed to measure changes in inductance, the labels may comprise iron oxide nanoparticles. Sensors 976 and 978 are used to measure changes in the local properties of the lateral flow strip 952 due to the flow of sample, nonspecific binding of labels and analyte-label complexes, or environmental factors. Measurements from sensors 976 and 978 of the compensation region can serve as a reference to improve the accuracy and reproducibility of measurements from sensors 972 and 974 of the test sensing region.

Sensors 970 may be added, removed, or modified in other embodiments depending on the specific requirements of the LFA-based biosensor of interest, including: the number of analytes being detected; the need for quantitative, semi-quantitative, or qualitative results readouts: or the sample matrix being tested. Pairs of sensors are not absolutely necessary, as only one sensor is needed to measure a signal; however, additional sensors can improve the performance of the testing device.

In different embodiments, other electronic components and circuitry such as analog to digital converters, operational amplifiers, antennas, and traces connecting these components may be assembled or etched onto the reel of backing structure 906 or connected from an external reader to the lateral flow strip 952 following the R2R manufacturing process.

Attention is now directed back to FIG. 10, which is a flowchart of an exemplary process of R2R manufacturing of the testing device, in accordance with some embodiments of the present invention. The testing device is as described herein, for example, with reference to FIG. 1 and FIG. 2. The process of R2R manufacturing of the testing device is visually depicted with reference to FIG. 9.

As used herein, the term roll and reel are interchangeable.

At 1002, a roll of a flexible planar element is unrolled. The roll of the flexible planar element includes multiple spaced apart electronic sensors disposed thereon. The flexible planar element may be implemented as a flexible PCB.

At 1004, an adhesive substrate is layered over a surface of the flexible planar element.

The adhesive substrate may be implemented as a roll of self-adhesive tape. The roll of self-adhesive tape is unrolled. The unrolled self-adhesive tape is layered over the surface of the flexible planar element.

Alternatively or additionally, the adhesive substrate may be implemented as glue. The glue may be layered, for example, by being sprayed by a sprayer, and/or deposited by a nozzle, and/or spread by an applicator.

At 1006, rolls of components for creating multiple lateral flow test elements are unrolled and layered.

At 1008, all of the components for creating the lateral flow test elements are laminated to the adhesive substrate to create a master assembly.

Processes described with reference to 1002, 1004, 1006, and/or 1008 may occur substantially simultaneously such that rolls are unrolled, layered, and laminated substantially in parallel.

At 1010, the master assembly is sliced into multiple slices. Each slice denotes a respective testing device for evaluating presence of an analyte in a sample applied thereon.

The slicing may be performed along the width of the master assembly such that there are an identical number of test and reference sensors on each testing device.

Each respective test device includes a planar element of the roll of flexible planar element, one or more electronic sensors of the roll of flexible planar element, and the adhesive substrate. The adhesive substrate is connected to a surface of the planar element and to a surface of the lateral flow test element for fixing the electronic sensor in a position facing the sensing region that includes analyte binding elements designed to bind to the analyte administered to the lateral flow test element.

Attention is now directed back to FIG. 12A and FIG. 12B, which show schematics of resonant magnetometers comprising exemplary planar elements implemented as PCBs and designed to connect to an LFA via an adhesive substrate, in accordance with some embodiments of the present invention. FIG. 12A depicts an all-in-one system comprising exemplary planar element 1202A. FIG. 12B depicts a two-part system comprising exemplary planar element 1202B and exemplary external reader 1270B.

Exemplary planar element 1202A is implemented as a single-use PCB. A long rectangular region 1204A (also referred to as a lateral flow assay contact region), which includes coils 1206A, outlines the position of the lateral flow assay, which is directly affixed to the PCB via a thin layer of adhesive, and/or may be manufactured using a R2R manufacturing approach, as described herein. PCB 1202A includes one or more of the following exemplary regions and components: a connection region 1210A for power supply, for example, a single use battery, a rechargeable battery, and/or a wireless charging module: low energy communication module 1212A for transmission (e.g., Bluetooth) of test results to another device (e.g., user's smart phone); analog to digital converter (ADC) 1214A; lateral flow assay contact region 1204A; coils 1206A, which may serve as a combination of test and reference coils in different embodiments.

Exemplary planar element 1202B is implemented as a single-use PCB which may be interchangeably connected to reusable exemplary external reader 1270B. A long rectangular region 1204B, which includes coils 1206B, outlines the position of the lateral flow assay, which is directly affixed to the PCB via a thin layer of adhesive, and/or may be manufactured using a R2R manufacturing approach, as described herein. PCB 1202B includes lateral flow assay contact region 1204B; coils 1206B which may serve as a combination of test and reference coils in different embodiments; and mating contact pads 1250B designed to form a connection to mating contact pins 1252B of an exemplary connector 1260B of exemplary reader 1270B. External reader 1270B includes one or more of the following exemplary regions and components: a connection region 1210B for power supply, for example, a rechargeable battery; a connector 1256B for power supply and/or data communication (e.g., USB-C port); low energy communication module 1212B for transmission (e.g., Bluetooth) of test results to another device (e.g., user's smart phone); mating contact pins 1252B, and ADC 1214B.

In some embodiments, the resonance magnetometer LFA readers described herein are based on the principle of resonant frequency. The coils may be implemented as a stack of four coils made of conductive material (e.g., copper, termed 4-layer coils), which are connected in series. Coils may alternatively or additionally be connected in parallel. Each coil may include more or fewer layers and/or turns. The specific design of the coils may be selected for optimization of the specific application.

The coils are subjected to an AC current, modulated by the inductive sensor, at a frequency matching the resonant frequency of the coils. This frequency is variable and a function of the coils' construction, geometry, and/or the electromagnetic fields in the environment surrounding the coils. The frequency at which the coils are resonating is then computationally converted into an inductance measurement and digitized, thereafter being passed on to a processor for signal collection, processing, and/or interpretation. Changes in the electromagnetic environment (e.g., magnets and/or foreign signals) and/or the coils' rapprochement and/or contact with physical objects (i.e., the lateral flow assay membrane) result in a change of the coils' resonant frequency, which is probed, for example, at a rate of 16 times per second at a resolution of 28 bits, or other values. A change in the coils' resonant frequency over time is monitored. The change provides information as to the status of the assay, such as to determine presence of the analyte therein.

The test pad membrane is brought into contact with the coils by the thin adhesive substrate, which is responsible for keeping the membrane in a fixed position for the duration of the test and also keeping the membrane as close to the coils as possible as the signal decays rapidly should the test be performed further away.

As discussed below in the “Examples” section, Inventors observed a signal change even when there were no nanoparticles travelling across the coils, through the test pad membrane. Inventors discovered that the liquid wetting the test pad membrane changes the material's properties as well as the environmental conditions directly in contact with the coils, which contributes somewhat to the inductance change. Inventors also observed that many of the samples and buffers running through LFAs are ionic, for example phosphate buffered saline has a salt ion concentration of over 150 mM, and the travel of these ions across the coils through the membrane is probed by the sensor, resulting in changes to the resonant frequency of the coils and the calculated inductance.

The run time of a lateral flow assay is extremely variable and can depend on a number of factors, for example, the materials chosen, the sample matrix's viscosity (e.g., saliva can flow slower than urine), the dimensions of the testing device, and factors like relative humidity in the casing and temperature. Based on Inventor's realization (based on experimentation) that results are reliably measured after the inductances of both coils have stabilized, after the stability has been determined, the relative values of the coils are analyzed to determine whether or not magnetic nanoparticles have accumulated in great enough amounts on the test line for the test to be deemed a positive result, or concentration to be quantified, for example, as described in additional detail in the “Examples” section below.

In inductive sensing embodiments, inventors realized (based on experimentation) that size of nanoparticles is significant, and is to be selected accordingly. After having run experiments using iron oxide nanoparticles of sizes ranging from 10 to 500 nanometers, Inventors discovered that superparamagnetic behavior was an important factor in achieving high-quality, reproducible results. For iron oxide or magnetite nanoparticles, superparamagnetic behavior is generally observed when the particle size is smaller than 16 nanometers, otherwise they are in the ferromagnetic domain. This occurs because in order to achieve a superparamagnetic regime the nanoparticle should ideally comprise a magnetic monodomain. This phenomenon, at least in the case of iron oxide and magnetite, can only occur for very specific sizes of nanoparticle, namely in the 10-14 nm range, although other. Larger clusters of superparamagnetic nanoparticles, for example with an average total diameter between 100-300 nm, show improved signal readouts due to the additive properties of the clusters' many monodomains. At a size above 16 nm, the iron oxide or magnetite nanoparticle is less likely to exhibit a magnetic monodomain, and each individual domain behaves differently because of temperature and environmental electromagnetic noise. This realization is in contrast, for example, to a supposition that the magnetic field of the coil (and thus the coil's calculated inductance) would be most affected when the mass of the magnetite or iron oxide is maximized.

In some embodiments, based on the ability to monitor flow of the liquid through the LFA (e.g., as described herein) and across the coils, and/or to detect when the flow of the liquid has stopped, a control line is not necessarily required.

Referring now back to FIG. 23 schematic 2304A corresponds to schematic 604A of FIG. 6, schematic 2304B corresponds to schematic 604B of FIG. 6, schematic 2304C corresponds to schematic 604C of FIG. 6, and schematic 2304D corresponds to schematic 604D of FIG. 6. The reader is invited to refer back to FIG. 6 while reviewing FIG. 24. The signal vs. time graphs 2308A-D show the progression of response curve 2310 for corresponding schematics 2304A-D, which show the difference between the signal of the reference coil and the test coil over time for each corresponding schematic 2304A-D.

Schematic 2304A depicts fluid sample 600 traveling from sample pad 260 to conjugate pad 262, where nanoparticles conjugated to detection molecules await. Since the sample has not wetted either of the reference and test regions over the reference sensor and test sensor, respectively, response curve 2310 remains relatively constant.

Schematic 2304B depicts fluid sample 600 wetting test pad 264 above reference sensor 272. The wetting of reference sensor 272 by sample 600 prior to test sensor 274 causes a rapid increase 2312 in response curve 2310, which is due to a substantially equivalent change in the local material properties (e.g., dielectric permittivity in the case of capacitive and inductive sensing) of the reference region measured by the reference sensor while the response of the test sensor remains relatively constant. Peak 2314 of response curve 2310 occurs once fluid sample 600 fully wets the portion of test pad 264 aligned and facing reference sensor 272.

Schematic 2304C depicts fluid sample 600 wetting test pad 264 above test sensor 274 and analyte-label complexes beginning to accumulate at test line 290 above test sensor 274. The wetting of test sensor 274 by fluid sample 600 causes a rapid decrease 2316 in response curve 2310, which negates signals arising due to the flow of fluid sample 600. Reference point 2318 of response curve 2310 occurs once fluid sample 600 fully wets the portion of test pad 264 aligned and facing test sensor 274. Analyte binding elements patterned on test pad 264 above test sensor 274 causes an accumulation of the analyte-label complexes whereas such complexes do not selectively bind above reference sensor 272. The accumulation of the complexes over test sensor 274 results in a proportional increase 2320 in response curve 2310.

Schematic 2304D depicts sample fluid 600 being absorbed by wick 266. As the bulk of sample fluid 600 passes through the LFA and is absorbed by wick 266, the change in response curve 2310 due to the accumulation of analyte-label complexes plateaus, which indicates the completion of the test. The plateau may arise due to the saturation of binding sites on test line 290 with analyte-label complexes or a decrease in the flow of sample fluid 600 as the bulk of the sample is absorbed by wick 266. A signal readout 2322 is measured as the difference in signal intensity of response curve 2310 between reference point 2318 and the completion of the test. In practical implementations of the testing device 250, the signal of the fully wetted sensors prior to analyte-label accumulation may not be equivalent to that of the sensors prior to wetting. Depending on the arrangement and configuration of test and reference sensors, the accumulation of analyte-label complexes may be indicated by an increase or decrease in the signal intensity of the response curve.

The signal readout may be used to determine the presence or amount (e.g., concentration) of analyte in a fluid sample.

Various embodiments and aspects of the present invention as delineated herein above and as claimed in the claims section below find experimental and/or calculated support in the following examples.

### EXAMPLES

Reference is now made to the following examples, which together with the above descriptions illustrate some embodiments of the present invention in a non-limiting fashion.

Inventors conducted experiments based on at least some implementations described herein.

A first experiment performed by Inventors is now described. Inventors evaluated signals from the magnetic inductance coil implementation of sensors of the testing device, as described herein.

Attention is now directed back to FIG. 13, which is a graph depicting a response curve from a reference coil 1302 and a response curve from a test coil 1304 over time, as measured by inventors, in accordance with some embodiments of the invention. Over the time interval, the membrane above the coils is progressively imbued with a fluid sample. Peaks 1314 and 1318 of response curves 1302 and 1304 correspond to the time at which the front of the fluid sample reached the center of each respective coil.

Time zero indicates when the sample was added to the LFA sample pad, and the information gathered from such graphs may inform as to the results of the assay. In the case of the data used to generate the graphs of FIG. 14, no nanoparticles were added to the test strip, only a buffer. Inventors discovered a signal change even when there were no nanoparticles travelling across the coils, though the nitrocellulose membrane. Inventors discovered that the liquid imbuing the nitrocellulose membrane changes the material's properties as well as the environmental conditions directly in contact with the coils, which contributes somewhat to the inductance change. Inventors also discovered that many of the samples and buffers running through LFAs are ionic, for example phosphate buffered saline has a salt ion concentration of over 150 mM, and the travel of these ions across the coils through the membrane is probed by the sensor, resulting in changes to the resonant frequency of the coils and the calculated inductance.

Attention is now directed back to FIG. 14, which is a graph depicting the phenomenon of ions flowing across coils creating changes to the resonant frequency of the coils and the calculated inductance, as measured by inventors, in accordance with some embodiments of the invention. Two tests were performed where initially the strip was wetted with H₂O. Thereafter, around the 2-minute mark, a drop (about 10 microliters (μL)) of PBS was added to the strip and allowed to travel. The resulting increase in signal is thanks to the movement of charge present in the buffer are plotted, where curve 1402 denotes inductance for H₂O over time, and curve 1404 denotes inductance of H₂O followed by the addition of PBS over time. An additional 10 μL of H₂O was added around 7 minutes to push the PBS through the membrane, which is indicated as a downwards trend for test 1404 beyond 7 minutes. The slight disturbances in both tests' signal readouts around minutes 2 and 7 are due to the 10 μL additions of additional fluid to the LFA in both tests.

Attention is now directed back to FIG. 15, which is a continuation of curves 1302 and 1304 from the graph of FIG. 13, extending for a longer amount of time, in accordance with some embodiments of the present invention. The test strips used in the experiment were run with the appropriate buffer. The strip used had no nanoparticles applied to it, as in FIG. 14. Signals stabilized about 50 minutes after the addition of the buffer, which is the time 1502 after which the inductance signals have substantially stabilized indicating nearly identical flow/wetting conditions over the test and reference sensing elements.

After time 1502, approximately at the 50-minute mark, the difference between the inductances of the test coil curve 1304 and reference coil curve 1302 are relatively small compared to the differences earlier in the test's lifecycle. Had there been nanoparticles and a corresponding analyte in this assay, the presence of nanoparticles at the test line would have increased the inductance of that coil relative to the reference, which represents a measurable signal output.

Attention is now directed back to FIG. 16, which is a graph computed by subtracting inductance of the test coil from the inductance of the reference coil after running the test for long enough that the inductances have stabilized, in accordance with some embodiments of the present invention. The graph of FIG. 16 includes a first line 1622 and a second line 1624 computed based on data collected over a respective first trial and second trial on different days, with 5 data points collected for each trial. The linear regression value (R²), which is greater than 0.99 for the first line 1622 and second line 1624 indicates reproducibility of the results.

Attention is now directed back to FIG. 17, which is a graph illustrating the difference in the dynamic response between positive curve 1702 and negative curve 1704 of the electromagnetic sensing (EMS) modality used in a testing device fabricated by Inventors, in accordance with some embodiments of the present invention. The distinction between curves 1702 and 1704 can be first seen at approximately 96 seconds after the application of sample to the test. At about this time, the dynamic signal exhibits an inflection point, after which the accumulation of nanoparticle labels at the test line can be resolved both electromagnetically and optically (in this case). As the nanoparticle labels accumulate at the test line over time, the inductance of the sensors measuring the positive test will continue to increase, whereas in the negative test the inductance will stabilize. This difference in behaviour, as well as the magnitude and rate of the signal's increase, can be used to determine diagnoses as well as quantify the amount of analyte present in the sample in conjunction with a calibration curve such as the one in FIG. 16.

Attention is now directed back to FIG. 18, which is a graph comparing electromagnetic data 1822 measured using embodiments described herein, to colorimetric data 1832 measured using standard optical approaches, illustrating the advantage electromagnetic sensing (EMS) has over optical sensing methods currently used in the lateral flow industry. Typically, the signal to noise ratio (SNR) of optical sensing technology drops below the limit of detection when there are between 100 million to one billion colorimetric labels at the test region, which in the case of the experiment used to obtain colorimetric data 1832, are colloidal gold nanoparticles with 15 nanometer (nm) diameters. Using existing approaches, the optical sensing modality, be it a smartphone camera, a photodiode, or another type of light capturing technology, is unable to resolve a colorimetric line consisting of less than approximately 100 million colorimetric labels on the lateral flow test. This means that in cases where the concentration of analyte is low but nonetheless present in the sample, there could be hundreds of millions of capture events at the test line but the assay will still report a false negative result.

A second experiment performed by Inventors is now described.

In this experiment, Inventors present a testing device prototype and data processing protocol for the measurement of magnetic IL-6 test strips based on embodiments described herein. Inventors demonstrate a limit of blank (LOB) of 0.41 ng/ml of IL-6 obtained by embodiments described herein.

The main objective of this experiment was to verify the sensitivity and dynamic range of the testing device prototype, based on embodiments described herein, with magnetic IL-6 lateral flow test strips. In addition to determining these key immunosensor characteristics, this experiment also served to investigate various data processing protocols for interpreting time series signal profiles collected from the testing device prototype, including different sensor compensation schemes and analytical techniques to improve the signal-to-noise ratio (SNR) of embodiments described herein.

Attention is now directed back to FIG. 19, which is a schematic of an exemplary inductive sensor configuration and immunosensor assembly used in the experiment for testing of magnetic IL-6 lateral flow strips, in accordance with some embodiments of the present invention. Schematic 1902 is an exploded view of the inductive sensor configuration and immunosensor assembly, which includes: magnetic IL-6 test strip 1952 comprising sample pad 1960, test pad 1964, and wick 1966, as well as control line 1991 and test line 1990; adhesive substrate 1954, and a flexible PCB backing 1956 comprising coil 0 (C0) 1978, coil 1 (C1) 1976, coil 2 (C2) 1974, and coil 3 (C3) 1972. Schematic 1904 depicts magnetic IL-6 test strip 1952 connected to flexible PCB backing 1956 via adhesive substrate 1954 and positioned such that C0 and C2 are aligned and facing control line 1991 and test line 1990, respectively.

To run the experiment, IL-6 samples were prepared by diluting IL-6 controls to their desired concentrations in 40 μL of running buffer (1×PBS, 0.5% Brij 98, 0.5% BSA). Magnetic nanoparticles (MNPs) were added to the IL-6 samples, followed by 5 μL of an anti-IL-6-biotin stock solution. Samples were incubated at room temperature for 5 minutes before being applied to test strips.

To collect measurements from the testing device, magnetic test strips were adhered to the flexible PCB backing with their control and test lines positioned over the sensor's coils as shown in schematic 1904 of FIG. 19. Once a strip was firmly adhered to the flexible PCB backing, the testing device prototype was activated using peripheral electronic hardware to begin collecting time series measurements. Following testing device activation, 50 μL of the desired sample preparation was applied to the sample pad of the adhered test strip and left to run for 15 minutes. Time series measurements were collected approximately 16 times per second throughout each 15-minute test period.

To compensate for changes in environmental conditions within and between tests, inductive measurements collected from the test-line coil, C2, were referenced against measurements that were simultaneously collected from adjacent coils, C1 and C3. Numbered coils are shown in schematic 1902 of FIG. 19. The reference coils were positioned over unpatterned segments of the test pad, which are not intended to selectively bind MNPs, as shown in schematic 1904 of FIG. 19. Two main compensation schemes were evaluated in this experiment, which consisted of subtracting the signals of the different reference coils from the test coil signal as follows: C3-C2 (C32) and C1-C2 (C12). A standard curve for each compensation scheme was fitted with a 4-parameter logistic regression model.

Attention is now directed back to FIG. 20, which includes standard curves presenting the effect of IL-6 concentration on signal readout of two inductive immunosensor compensation schemes used in experiment that evaluated the testing of magnetic IL-6 lateral flow strips, in accordance with some embodiments of the present invention. Curves 2022A and 2022B are standard curves for the C32 and C12 compensation schemes, respectively. Signal readouts of standard curves were measured at approximately 13000 data points (i.e., about 13 minutes after sample administration).

All error bars indicate standard deviations of tests run in triplicate. The operating range (i.e., the difference between each curve's upper and lower limits) of the C32 compensation scheme as depicted in curve 2022A is 1.23 times greater than that of the C12 scheme as depicted in curve 2022B. Moreover, the average standard deviation of C32 was only 1.05 times greater than that of C12, which suggests that signal magnitude (i.e., operating range) is not directly proportional to measurement variations observed across the two compensation schemes.

Measurement variations are likely due to human error during the testing process. For example, in each compensation scheme, the triplicates run at 30 ng/ml had an abnormally large standard deviation, which could have been caused by strip positioning or placement errors during testing that led to variations in the alignment or adhesive contact of the test line aligned and facing C2.

Compensation schemes C32 and C12 both showed LODs of 3 ng/ml of IL-6, while they showed different LOBs of 0.41 and 0.53 ng/ml of IL-6, respectively. LOB approximations were calculated using the 4-parameter logistic regression model and 3.3 times the standard deviation at 0 ng/ml for each standard curve. This experiment ultimately shows that the Inventors' testing device prototype has the potential to produce highly sensitive tests for the detection and quantitation of health-related biomarkers in point-of-care settings.

Each test conducted with the Inventors' testing device prototype, based on embodiments described herein, generates time series profiles of inductive measurements from the planar coils of the flexible PCB backing 1956 adhered to the test pad 1964.

Attention is now directed back to FIG. 21, which includes time series signal profiles of two inductive immunosensor compensation schemes for a test run at 10 ng/ml of IL-6, of the IL-6 experiment, in accordance with some embodiments of the present invention. Curves 2108A and 2108B are exemplary time series signal profiles for the C32 and C12 compensation schemes, respectively. The profiles of both these compensation schemes have distinct and localized patterns referred to as features, which appear regardless of the concentration of analyte run through each test. Each feature is localized to a single point from the time series profile, known as a feature point. Feature points are used as references to standardize measurements across different tests, analogous to taring a scale. Any tests that do not contain these feature points could be considered invalid, although none of the tests in this experiment deviated from their expected profiles.

Based on the method of data processing presented here, each compensation profile has two feature points: a global extremum and a zero point. As indicated by triangles 2114A and 2114B in FIG. 21, the global extrema for the C32 and C21 schemes are minima and maxima, respectively. The zero point is more difficult to define, but is generally considered to be the first local extremum after a profile's global extremum, and is indicated by circles 2118A for C32 and 2118B for C21 in FIG. 21. Signal readouts 2122A and 2122B are measured as the difference in signal magnitude between the zero point and the end of the time series signal.

Attention is now directed back to FIG. 22, which depicts averaged truncated time series signal profiles of two inductive immunosensor compensation schemes for all of the tests run in the IL-6 experiment, in accordance with some embodiments of the present invention. Graphs 2208A and 2208B depict time series signal profiles truncated at their zero points for the C32 and C12 compensation schemes, respectively. Time series signal profiles 2200A and B, 2201A and B, 2211A and B, 2213A and B, 2221A and B, 2223A and B, 2233A and B show tests run at 0, 0.1, 1, 3, 10, 30, and 300 ng/ml, respectively. Each profile is an average of triplicates. C32 profiles shown in 2208A show a slight dip over the first 1-2 minutes of tests run at lower concentrations of IL-6 (0-10 ng/ml). Otherwise, most of the profiles in FIG. 22 exhibit a characteristic logarithmic trend, which is reflective of MNPs accumulating at the test line.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant LFAs will be developed and the scope of the term LFA is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A testing device for evaluation of at least one analyte, comprising:
a planar element;
at least one electronic sensor disposed on the planar element for sensing at least one sensing region of a lateral flow test element for evaluation of the at least one analyte; and
an adhesive substrate connected to a surface of the planar element and set to connect to a surface of the lateral flow test element.

2. (canceled)

3. The testing device of claim 1, wherein the adhesive substrate comprises a first and second adhesive opposing surfaces, the first adhesive surface connected to the surface of the planar element, the second adhesive surface set to connect to the surface of the lateral flow test element.

4. The testing device of claim 1, wherein the adhesive substrate is selected from a group consisting of: glue, tape, a self-adhesive, and a spray-on adhesive.

5. The testing device of claim 1, further comprising the lateral flow test element.

6. The testing device of claim 1, wherein the adhesive substrate places the at least one sensing region(s) proximate to the at least one electronic sensor.

7. (canceled)

8. The testing device of claim 1, wherein the adhesive substrate is sandwiched between the surface of the planar element and the surface of the lateral flow test element.

9. (canceled)

10. The testing device of claim 1, wherein the adhesive substrate is disposed at least over the at least one electronic sensor and the at least one sensing region, wherein a region of the lateral test flow element that includes the at least one sensing region is aligned with the at least one electronic sensor.

11. (canceled)

12. The testing device of claim 1, wherein the planar element replaces a backing card of the lateral flow test element that otherwise serves as a physical support component of the lateral flow test element.

13. (canceled)

14. The testing device of claim 1, wherein the surface of the planar element has a size of at least the size of the surface of the lateral flow test element.

15. The testing device of claim 1, wherein the planar element comprises a printed circuit board (PCB), wherein the at least one electronic sensor is at least one of: connected to and integrated within the PCB.

16. The testing device of claim 1, further comprising at least one second electronic sensor disposed on the planar element for sensing at least one reference region of the lateral flow test that does not substantially include analyte binding elements, wherein the at least one analyte administered to the lateral flow test element flows across the at least one reference region without substantially selectively binding thereto.

17. (canceled)

18. The testing device of claim 1, wherein the at least one sensing region comprises a testing region and a reference region distinct from the testing region, wherein the at least one electronic sensor comprises a test electronic sensor located on the planar element for sensing the testing region, and a reference electronic sensor located on the planar element for sensing the reference region.

19. The testing device of claim 1, wherein the at least one sensing region includes analyte binding elements designed to bind to the at least one analyte corresponding to:
infectious disease biomarkers for evaluation of presence of the infectious disease in a user;
health-related biomarkers for evaluation of relevant health conditions in a user; or
food-related biomarkers for evaluation of food safety by a user.

20.-22. (canceled)

23. The testing device of claim 1, wherein the at least one electronic sensor is designed to generate electromagnetic fields that follow field lines that originate from the at least one electronic sensor, pass through the adhesive substrate, pass through the at least one sensing region, pass back through the adhesive substrate, and back to the at least one electronic sensor.

24. The testing device of claim 1, wherein the at least one electronic sensor comprises at least one capacitive sensor for capacitive sensing.

25. (canceled)

26. The testing device of claim 1, wherein the at least one electronic sensor comprises at least one inductive sensor for inductive sensing.

27. The testing device of claim 26, wherein each inductive sensor comprises at least one planar coil with at least one layer.

28. The testing device of claim 26, further comprising circuitry configured to activate the at least one inductive sensor at a resonant frequency thereof.

29. The testing device of claim 28, further comprising a controller operating at a sampling frequency and configured to measure changes in the resonant frequency of the at least one inductive sensor indicating binding of the at least one analyte to the at least one sensing region.

30. The testing device of claim 29, wherein the resonant frequency of the at least one inductive sensor is in a range of 0.001-40 megahertz (MHZ).

31. The testing device of claim 26, wherein a label selected to exhibit superparamagnetic properties is used to identify the presence of the at least one analyte.

32. The testing device of claim 1, further comprising a transceiver configured to transmit signals sensed by the at least one electronic sensor to an external computing device for analysis of the signals for measuring presence of the at least one analyte.

33. The testing device of claim 1, wherein the testing device is implemented as a single-use device designed to be disposed of after a liquid sample is applied to the lateral flow test element, the liquid sample flows through the lateral flow test element by capillary action and reaches a wick, and the at least one analyte when present in the solution binds to analyte binding elements of the at least one sensing region.

34.-50. (canceled)
